(19) United States
(12) Patent Application Publication
(10) Pub. No.: US 2024/0066147 A1
(43) Pub. Date: Feb. 29, 2024

(54) AAV-MEDIATED EXPRESSION USING A SYNTHETIC PROMOTER AND ENHANCER

(71) Applicant: University of Iowa Research Foundation, Iowa City, IA (US)

(72) Inventors: John F. Engelhardt, Iowa City, IA (US); Ziying Yan, Iowa City, IA (US)

(21) Appl. No.: 18/139,661

(22) Filed: Apr. 26, 2023

Related U.S. Application Data
(62) Division of application No. 16/082,767, filed on Sep. 6, 2018, now Pat. No. 11,684,679.
(60) Provisional application No. 62/304,656, filed on Mar. 7, 2016.

(51) Int. Cl. A61K 48/00; A61K 47/55; C12N 15/86

(52) U.S. Cl. CPC A61K 48/0066; A61K 47/55; C12N 15/86; C12N 2750/14041; C12N 2750/14043; C12N 2750/14143

(57) ABSTRACT

An isolated recombinant parvovirus vector comprising a synthetic enhancer comprising plurality of enhancer sequences operably linked to a promoter, and methods of using the vector, are provided.

### CROSS-REFERENCE TO RELATED APPLICATIONS

This application is a division of U.S. application Ser. No. 16/082,767, filed on Sep. 6, 2018, which application Is a U.S. National Stage Filing under 35 U.S.C. 371 from International Application No. PCT/US2017/021124, filed on Mar. 7, 2017, and published as WO 2017/155973 on Sep. 14, 2017, which application claims the benefit of the filing date of U.S. Application Ser. No. 62/304,656, filed on Mar. 7, 2016, the disclosures of which are incorporated by reference herein.

### STATEMENT OF GOVERNMENT RIGHTS

The invention was made with government support under grant HL108902 awarded by the National Institutes of Health. The Government has certain rights in the invention.

### BACKGROUND

Cystic fibrosis (CF) is a lethal, autosomal-recessive disorder that affects at least 30,000 people in the U.S. alone (O'Sullivan et al., 2009). The genetic basis of CF is mutation of a single gene that encodes the cystic fibrosis transmembrane conductance regulator (CFTR) (Riordan et al., 1989; Rommens et al., 1989). This results in a defective CFTR protein and consequent abnormalities in the transport of electrolytes and fluids in multiple organs (Welsh, 1990; Rowe et al., 2005). The most life-threatening outcome is CF pulmonary disease, which is characterized by viscous mucous secretions and chronic bacterial infections (Welsh, 1990). With improvement in patient care and advances in pharmacologic therapies for CF, the lifespan of CF patients has steadily been extended over the past decades; however, the quality of life for CF patients remains poor, and medications that alleviate pulmonary complications are expensive and efficacious only in select patients. Since lung disease is the major cause of mortality in CF patients and the genetic basis is a single-gene defect, gene therapy for CF lung disease has the potential to cure all CF patients, regardless of their CFTR mutation. Thus, clinical trials for CF lung gene therapy were initiated in the mid-1990s. However, all trials to date have been unsuccessful (Sumner-Jones et al., 2010). The underlying reason is that the vectors available for gene transfer to the human airway epithelium (HAE) are inefficient (Mueller & Flute, 2008; Griesenbach & Alton, 2009; Griesenbach et al., 2010).

Adeno-associated virus (AAV), a member of the human parvovirus family, is a non-pathogenic virus that depends on helper viruses for its replication. For this reason, rAAV vectors are among the most frequently used in gene therapy pre-clinical studies and clinical trials (Carter, 2005; Wu et al., 2006; Daya & Berns, 2008). Indeed, CF lung disease clinical trials with rAAV2 demonstrated both a good safety profile and long persistence of the viral genome in airway tissue (as assessed by biopsy) relative to other gene transfer agents (such as recombinant adenovirus). Nevertheless, gene transfer failed to improve lung function in CF patients because transcription of the rAAV vector-derived CFTR mRNA was not detected (Flotte, 2001; Aitken et al., 2001; Wagner et al., 2002; Moss et al., 2007; Duan et al., 2000). These observations are consistent with later studies on rAAV transduction using an in vitro model of the polarized HAE, in which the cells are grown at an air-liquid interface (ALI) (Flotte, 2001; Duan et al., 1998). The poor efficiency of rAAV2 as a vector for CFTR expression in the HAE is largely due to two major barriers: 1) inefficient post-entry processing of the virus, and 2) the limited packaging capacity of rAAV.

The initial preclinical studies with rAAV2-CFTR that supported the first clinical trial in CF patients were performed in rhesus monkeys. These studies demonstrated that viral DNA and transgene-derived CFTR mRNA persisted in the lung for long periods following rAAV2-mediated CFTR gene transfer (Conrad et al., 1996). However, later studies comparing the efficiency of rAAV2 transduction between human and rhesus monkey airway epithelial ALI cultures demonstrated that the tropism of rAAV2 for apical transduction was significantly higher in the rhesus monkeys cultures than in their human counterparts (Liu et al., 2007), likely due to species-specific differences in the AAV2 receptors and co-receptors that exist on the apical surface. In studies of polarized HAE, the majority of AAV2 virions were internalized following apical infection, but accumulated in the cytoplasm rather than entering the nucleus (Duan et al., 2000; Ding et al., 2005). One obstacle to the intracellular trafficking required for productive viral transduction is the ubiquitin-proteasome pathway (Duan et al., 2000; Yan et al., 2002); transient inhibition of proteasome activity dramatically enhances transduction (700-fold) of rAAV2-luciferase vectors from the apical surface by facilitating translocation of the vector to the nucleus (Yan et al., 2006). However, the application of proteasome inhibitors to enhance transduction efficiency of rAAV-CFTR vectors only marginally improves CFTR expression, most likely due to the low activity of the short promoter used in the rAAV-CFTR vectors (Zhang et al., 2004). The open reading frame (ORF) of the CFTR gene is 4.443 kb, and thus approaches the size of the 4.679 kb AAV genome. Although the AAV capsid can accommodate content in excess of its native DNA genome, its maximum packaging capacity is approximately 5.0 kb (Dong et al., 1996), and transgene expression from vectors exceeding this limit result in significantly reduced function (Wu et al., 1993). Given the requirements for 300 bp of cis-elements from the AAV genome (two ITR sequences at the termini) and the 4,443 bp CFTR coding sequence, there is little space left in the vector genome (257 bp) for a strong promoter and polyadenylation signal. Thus, the first-generation rAAV-CFTR vector (AV2.tgCF) that was tested in clinical trials, relied on the cryptic promoter activity of the AAV2 ITR to drive transcription of the full-length CFTR cDNA with a synthetic polyadenylation signal (Flotte et al., 1993; Aitken et al., 2003).

More recently, a rAAV vector, AV2.tg83-CFTR was developed, which uses an 83-bp synthetic promoter (tg83) (Zhang et al., 2004) to improve expression of the full-length human CFTR cDNA. The genome of this vector is 4.95 kb in size. Although this vector produced a 3-fold increase in cAMP-mediated Cl⁻ currents in CF HAE ALI cultures relative to AV2.tgCF, this level of expression remained suboptimal for application in CF gene therapy. Other groups have attempted to use a CFTR minigene to create space for incorporating a better promoter into the rAAV vectors; this seemed justified based on earlier studies of CFTR gene function and structure indicating that the deletion of short, nonessential sequences from the C-terminus and regulatory domain (R-domain) had only minimal effects on the chloride channel function of CFTR (Zhang et al., 1998). One widely used CFTR minigene is CFTRΔR, which lacks 156 bp encoding 52 amino acid residues (708-759) at the N-terminus of the R-domain. Gene transfer with a recombinant adenoviral vector encoding CFTRΔR in CF HAE ALI cultures demonstrated that this transgene retains at least 80% of the transepithelial Cl⁻ transport supported by full-length CFTR (Ostedgaard et al., 2002). In addition, the expression of CFTRΔR in CFTR^{-/-} knockout mice rescued the lethal intestinal phenotype (Ostedgaard et al., 2011). This 156 bp deletion made it possible to package a rAAV CFTR expression vector 4.94 kb in length, with expression driven by a minimal CMV promoter (173 bp), into an AAV5 capsid (Ostedgaard et al., 2005). Additional efforts were aimed at developing AAV variant vectors of higher apical tropism, through directed evolution of the AAV capsid in polarized HAE ALI cultures (Li et al., 2009). However, these rAAV vectors did not provide efficient CFTR expression because the minimal CMV promoter did not function well in fully differentiated airway epithelia.

### SUMMARY

To circumvent the size limitation of the promoter in a recombinant adeno-associated viral (rAAV) vector that can be used to express certain transgenes, a set of 100-mer synthetic enhancer elements, composed of ten 10 bp repeats, were screened for the ability to augment CFTR transgene expression from a short 83 bp synthetic promoter in the context of a rAAV vector for application in cystic fibrosis (CF) gene therapy. Screening for the effectiveness of synthetic enhancers to augment transgene expression was conducted in a stepwise fashion—in plasmids without AAV sequences, proviral vectors in the form of plasmids with AAV sequences, and rAAV vectors. Both plasmid transfection and viral vector transduction in cultured cell lines and whole animals in vivo were evaluated. Initial studies assessing transcriptional activity in monolayer (non-polarized) cultures of human airway cell lines and primary ferret airway cells revealed that three of these synthetic enhancers (F1, F5, and F10) significantly promoted transcription of a luciferase transgene in the context of plasmid transfection. Further analysis in polarized cultures of human and ferret airway epithelia at an air-liquid interface (ALI), as well as in the ferret airway in vivo, demonstrated that the F5 enhancer produced the highest level of transgene expression in the context of an AAV vector. Furthermore, it was demonstrated that increasing the size of the viral genome from 4.94 to 5.04 kb did not significantly affect particle yield of the vectors, but dramatically reduced the functionality of rAAV-CFTR vectors because of small terminal deletions that extended into the CFTR expression cassette of the 5.04 kb oversized genome. Since rAAV-CFTR vectors greater than 5 kb in size are dramatically impaired with respect to vector efficacy, a shortened ferret CFTR minigene with a 159 bp deletion in the R-domain was utilizing to construct a rAAV vector (AV2/2.F5tg83-fCFTRΔR). This vector yielded an about 17-fold increase in expression of CFTR and significantly improved Cl⁻ currents in CF ALI cultures. This small enhancer/promoter combination may have broad utility for rAAV-mediated gene therapy, e.g., CF gene therapy, to the airway.

The disclosure provides a recombinant vector such as a parvovirus vector, e.g., a recombinant adeno-associated virus (rAAV) vector or a bocavirus (BoV), such as a human BoV, vector, comprising a synthetic enhancer having a plurality of synthetic enhancer sequences operably linked to a promoter, e.g., a synthetic promoter. In one embodiment, each of the plurality of enhancers has the same sequence. In one embodiment, at least 2 of the plurality of enhancers have a different sequence. In one embodiment, the synthetic enhancer is formed of different enhancer sequences, where each unique sequence may be represented once or more than once, and if more than once, may be in tandem or interspersed with other (different) enhancer sequences. For example, the synthetic enhancer may have five different enhancer sequences, each represented twice in the synthetic enhancer, and the repeated sequences may be in tandem (or not). In one embodiment, at least one of the enhancer sequences has a TP53 binding site. In one embodiment, at least one of the enhancer sequences has a CREB binding site. In one embodiment, at least one of the enhancer sequences has a NRF-1 binding site (CATGCGCAG). In one embodiment, plurality has a combination of one or more TP53 binding sites, one or more NRF-1 binding sites, and/or one or more CREB, e.g., CREB7, binding sites. In one embodiment, the enhancer sequence has a binding site shown in one of FIGS. 8A-8C. In one embodiment, the plurality has 2 up to 20 distinct synthetic enhancer sequences In one embodiment, at least one of the enhancer sequences has no more than 15 bp. In one embodiment, the plurality is up to about 150 nucleotides in length, e.g., from about 20, 30, 40, 50, 60, 70, 80, 90, 100, 110, 120, 130, or 140 nucleotides in length. In one embodiment, the synthetic enhancer comprises F1, F5 or F10. In one embodiment, the enhancer has at least 80%, 85%, 90%, 92%, 95%, 98% or 99% nucleotide sequence identity to F1, F5 or F10. In one embodiment, the linked promoter is a synthetic promoter. In one embodiment, the promoter is tg83. In one embodiment, the promoter is an AAV promoter. In one embodiment, the promoter is a heterologous promoter, e.g., from a different virus or from a mammalian genome. In one embodiment, the promoter is operably linked to an open reading frame, e.g., a heterologous open reading frame. In one embodiment, the open reading frame encodes a prophylactic or a therapeutic gene product, e.g., cystic fibrosis transmembrane conductance regulator, α-antitrypsin, β-globin, γ-globin, tyrosine hydroxylase, glucocerebrosidase, aryl sulfatase A, factor VIII, dystrophin or erythropoietin. In one embodiment, the combination of the plurality of enhancer sequences and the promoter is no more than 300 nucleotides in length, e.g., no more than 125, 150, 175, 200, 250, or 275 nucleotides in length. In one embodiment, the combination of the plurality of enhancer sequences and the promoter is less than 500 nucleotides in length. In one embodiment, the vector is a parvovirus vector such as a rAAV vector, e.g., an AAV1, AAV2, AAV3, AAV4, AAV5, AAV6, AAV7, AAV8, or AAV9 vector, or a human bocavirus vector, e.g., HBoV1, HBoV2, HBoV3 or HBoV4, or an evolved AAV or HBoV vector that adapts a unique tropism, e.g., optionally one with slightly altered capsid sequences from known serotypes

This disclosure also relates to an approach to screen tissue-specific as well as ubiquitous synthetic promoter/enhancer combinations in a step-wise fashion, in plasmids, proviral vectors, and rAAV vectors, which can be used in the application of rAAV gene therapy for the delivery of large transgene cassette. Examples of use include but are not limited to express 4.3 kb B-domain deleted Factor-VIII in muscle and/or liver for hemophilia A, or to deliver the 4.2 kb the gene-editing tool of Streptococcus pyogenes (SpCas9) and a chimeric sgRNA together in any desired tissue and organ in vivo.

Further provided are methods of using the recombinant parvovirus vector to infect cells, e.g., mammalian cells such as ferret, canine, feline, bovine, equine, caprine, or porcine cells, or primate cells, e.g., human cells, for example, administering a composition comprising the recombinant parvovirus vector to a mammal. For example, the recombinant parvovirus genome may include an expression cassette encoding a heterologous gene product, e.g., which is a therapeutic protein such as cystic fibrosis transmembrane conductance regulator, α-antitrypsin, β-globin, γ-globin, tyrosine hydroxylase, glucocerebrosidase, aryl sulfatase A, factor VIII, dystrophin, erythropoietin, alpha 1-antitrypsin, surfactant protein SP-D, SP-A or SP-C, erythropoietin, or a cytokine, e.g., IFN-alpha, IFNγ, TNF, IL-1, IL-17, or IL-6, or a prophylactic protein that is an antigen such as viral, bacterial, tumor or fungal antigen, or a neutralizing antibody or a fragment thereof that targets an epitope of an antigen such as one from a human respiratory virus, e.g., influenza virus or RSV including but not limited to HBoV protein, influenza virus protein, RSV protein, or SARS protein.

### BRIEF DESCRIPTION OF THE FIGURES

FIGS. 1A-1D. Effectiveness of synthetic oligonucleotide enhancers in augmenting activity of tg83-directed luciferase reporter plasmids in monolayer cultures. A) Schematic structure of the reporter vectors used to screen the enhancer library. The transcriptional motifs of the synthetic tg83 promoter are indicated. (B-D) Reporter activity in monolayer cultures of human airway cell lines (B) A549 and (C) IB3, and (D) ferret primary airway cells, following transfection with the indicated plasmids. Luciferase assays were conducted 24 hours post-transfection. Data represent the mean (+/−SEM, N=3) relative luciferase activity of each transfection normalized to that of the enhancer-less vector pGL3-tg83luc, whose value in each cell-type tested was set to 1.

FIGS. 2A-2F. Effectiveness of enhancers F1. F5 and F10 in augmenting activity of the tg83 promoter in the context of proviral plasmids and rAAV. A) Effect of the AAV2 ITR on transcription from the tg83 promoter, as evaluated following transfection of A549 and primary airway ferret cells with pGL3-tg83 or pAV2-tg83luc. Data represent the mean (+/−SEM, N=3) relative luciferase activity (RLU) 24 hours post-transfection. B) Effectiveness of enhancers on transcription following transfection of A549 and primary ferret airway cells with the indicated AAV2 proviral plasmids. Data represent the mean (+/−SEM, N=3) relative luciferase activity of each transfection, normalized to the enhancer-less vector pAV2-tg83luc (set to 1 for each cell type), at 24 hours post-transfection. C) Effectiveness of enhancers on transcription following infection of A549 cells with the indicated rAAV2 vectors at 24 hours post-Infection. Data represent the mean (+/−SEM, N=3) relative luciferase activity for each infection, normalized to the enhancer-less vector pAV2-tg83luc (set to 1). (D and E) Effectiveness of enhancers on transcription following basolateral infection of polarized (D) human and (E) ferret airway epithelia infected with 2×10¹⁰ DRP of the indicated rAAV2 vectors. Data represent the mean (+/−SEM, N=4) relative luciferase activity (RLU) for each condition at 2 days post-infection. (F) Effectiveness of enhancers on transcription in lung and tracheal tissue following infection of 5 day-old ferret pups with 2×10¹¹ DRP of AV2/1.F5tg83luc or AV2/1.F10tg83luc, in the presence of proteasome inhibitors. Luciferase activity was measured 8 days post-infection. Data represent the mean (+/−SEM, N=4) relative luciferase activity (RLU/μg protein).

FIGS. 3A-3C. Impact of rAAV-CFTR construct size on restoration of CFTR chloride currents in polarized CF airway epithelium. A) Schematic Illustration of structures of rAAV2 vectors of distinct sizes that encode the full-length ferret CFTR open reading frame (ORF) and R domain-deleted variants, under the control of the same transcriptional elements: the 83 bp synthetic promoter (tg83), a 62 bp synthetic polyadenylation signal (pA), a 17 bp 5′ untranslated region (UTR), and a 9 bp 3′ UTR. The ORF of full-length ferret CFTR (fCFTR) is 4455 bp. The 99 bp 3×HA tag was inserted between amino acid residues S900 and I901, bringing fCFTR(HA) to 4554 bp. The fCFTRΔR has a shortened ORF (4,296 bp); 53 amino acid residues (I708-I760, 159 bp) are deleted from the R-domain. fCFTRΔR(HA) is 4395 bp in length, having a 99 bp HA-tag insertion and a 159 bp deletion in the R-domain. The functionalities of these vectors on rescue of CFTR specific Cl⁻ transport (reflecting by transepithelial short-circuit currents (Isc)) were compared in differentiated CF HAE ALI cultures, following infection at 10¹¹ DRP per Millicell insert (MOI of about 10⁵ DRP/cell in the presence of the proteasome inhibitors LLnL (10 μM) and doxorubicin (2 μM). CF HAE ALI cultures were generated from a conditionally transformed human CF airway cell line (CuFl8; genotype ΔF508/ΔF508). B) Typical traces of the Isc changes from CF ALI cultures, infected Indicated AAV-CFTR vectors, following the sequential addition of various inhibitors and agonists. Amiloride and DIDS were used to block ENaC-mediated sodium currents and non-CFTR chloride channels prior to addition of cAMP agonist (forskolin and IBMX), and GlyH101 was used to block CFTR-specific currents. ΔIsc (IBMX & Forsk) reflects the activation of CFTR-mediated chloride currents following induction with cAMP agonist, and ΔIsc (GlyH101) reflects the inhibition of CFTR-mediated chloride currents following addition of GlyH101. C) Effects of vector size on rescue of chloride Isc currents. rAAV-CFTR vectors were increased by about 100 bp increments. Shown are the ΔIsc (IBMX & Forsk) and ΔIsc (GlyH101) responses, indicating the magnitude of CFTR-mediated chloride transport following basolateral infection CF HAE ALI, as described in (B). CFTR currents generated from primary non-CF HAE (N=14) are provided for comparison. Data represent the mean (+/−SEM) for N=3 independent Millicell inserts.

FIGS. 4A-4B. Analyses of integrity of the viral genome by denaturing gel electrophoresis and slot blot analysis. A) Viral DNA was extracted from 10⁹ DRP of the indicated AAV-CFTR vectors, resolved on a 0.9% alkaline agarose gel, and transferred to a Nylon membrane. Southern blotting was performed with a ³²P-labeled CFTR probe to visualize the viral DNA. B) To assess potential deletion that may have occurred at the termini of positive and minus strand viral genomes, 3.33×10⁸ DRP of each virus (titer determined by TaqMan PCR with probe/primer set against fCFTR cDNA) were loaded in triplicate onto a Slot-Dot® SF Module (Bio-Rad, Hercules, CA) fitted with a Nylon membrane. 3-fold serial dilution of proviral plasmid (3×10⁹ to 3.7×10⁷ copies) was also loaded for generating the standard curves for quantitation. The blots were probed with ³²P-labeled oligonucleotides against the tg83 promoter, CFTR cDNA, or polyA. (−) and (+) represent the probes hybridizing to the minus and positive strand of the single stranded rAAV genome. Hybridization was first conducted with the set of probes that hybridize to the minus strand, and then reprobed with the set of oligonucleotides that hybridize to the positive strand. The number of viral genome copies detected by each probe was determined (mean+/−S.E.M, N=3) based on measurement of the signal density using NIH ImageJ and comparison to standard curves.

FIG. 5. Effects of the F5 enhancer on CFTR currents generated by CF HAE following infection with rAAV vectors. CF HAE ALI were infected with AV2/2.tg83-fCFTRΔR or AV2/2.F5tg83-fCFTRΔR, at the indicated MOIs, from the apical or basolateral surface. Proteasome inhibitors were co-administrated during the 18 hours infection period. Isc measurements of the infected ALI cultures were conducted at 2 weeks post-infection. The mean (+/−SEM) ΔIsc (IBMX & Forsk) and ΔIsc (GlyH101) are shown with the N for independent transwells assays indicated. Mock-infected CF and non-CF HAE cultures are shown for reference.

FIGS. 6A-6B. Effects of the F5 enhancer on CFTR currents and tg83-directed CFTR transcription following infection with rAAV vectors. CF HAE ALI were infected with AV2/2.tg83-fCFTRΔR or AV2/2.F5tg83-fCFTRΔR at an MOI of 2×10⁴ DRP/cell from the basolateral surface, in the presence of proteasome inhibitors. A) Isc was measured in the infected ALI cultures at 3 and 10 days post-infection. ΔIsc (IBMX & Forsk) and ΔIsc (GlyH101) values are presented. B) The abundance of vector-derived CFTR mRNAs in cultures evaluated in Panel a, as determined using RS-PCR and normalized to GAPDH transcripts in each sample. Data represent the mean (+/−SEM) for N=3 independent transwells in each panel.

FIGS. 7A-7B. 5 day old ferrets were systemically infected with 2×10¹¹ DRP AAV2/9F5tg83luc or AAV2/9F10tg83luc via jugular vein injection. Animals were euthanized 8 day post-infection, snap tissues from different organs were harvested and homogenized in reporter lysis buffer (Promega) for luciferase assays. A) Data compare the luciferase expression from the infections of AAV2/9F5tg83luc or AAV2/9F10tg83luc when the luciferase expression from F5tg83 promoter was arbitrarily set as 100 in each tissue. B) Values represent the (mean+SEM, n=3) relative luciferase activity (RLU/μg protein).

FIGS. 8A-8W. A-E) Binding sites in F5 which may be employed to prepare synthetic enhancers as described herein. F-P) Binding sites in F10 which may be employed to prepare synthetic enhancers as described herein. Q-W) Binding sites in F5tg83, which may be employed to prepare synthetic enhancers or promoter as described herein. SEQ ID NOs: 31-60 and 63-68.

FIGS. 9A-D. Gene transfer efficiency of AV.F5Tg83-hCFTRΔR to the ferret trachea and lung. Three day old ferrets were infected with a 100 μL volume of 6×10¹¹ DRP of AV.F5Tg83-hCFTRΔR in 500 μM doxorubicin. Non-infected animals were given an equal volume of vehicle with doxorubicin. At 10 days post-infections the entire lung and trachea were harvested and snap frozen in liquid nitrogen. Tissue was pulverized and mRNA and cDNA generated for Q-PCR of human and ferret CFTR. (A and B) Copies of hCFTR and fCFTR mRNA in the (A) trachea and (B) lung. Copy number was determined using a standard curve generated from serial dilutions of plasmid CFTR cDNA for each species. (C and D) Ratio of transgene-derived hCFTR to endogenous fCFTR mRNA. C1-C3 represent animals in the mock-infected group and A1-A3 represent animals in the AAV-infected group. The average is also shown for the three AAV-infected animals. The dashed line represents endogenous levels of CFTR (ratio=1). Data depicts the mean+/−SEM for N=3 animals in each group.

FIGS. 10A-D. AV.F5Tg83-hCFTRΔR effectively transduces the mature ferret airways. The lungs of 1 month old ferrets (N=3) were transduced with 7.5×10¹² DRP of AV.F5Tg83-hCFTRΔR harboring the hCFTRΔR cDNA in a 500 μL volume of PBS in the presence of 250 μM doxorubicin. A mock-infected control animal (N=1) received 500 μL PBS with no vector in the presence of 250 μM doxorubicin. Vector was delivered to the lung with a PennCentury microsprayer through tracheal intubation. Nasal delivery in the same animals was also performed using 100 μL containing 1.5×10¹² DRP with 250 μM doxorubicin by instillation of fluid. Mock-infected nasal delivery received PBS with 250 μM doxorubicin. At 12 days following infection, the lung lobes were harvested separately along with the trachea, carina, and nasal turbinates with surrounding adventitia. The tissues were snap frozen and pulverized samples were processed separately for mRNA and DNA. A) TaqMan RNA-specific PCR (RS-PCR) for human CFTR mRNA and endogenous ferret GAPDH mRNA for vector and mock treated animals. Results show the ratio of hCFTR/fGAPDH mRNA. B) TaqMan RS-PCR for endogenous ferret CFTR mRNA and endogenous ferret GAPDH mRNA for vector and mock treated animals. Results show the ratio of fCFTR/fGAPDH mRNA. C) TaqMan Q-PCR for the number vector genomes in each sample per 100 ng DNA. D) The ratio of mRNA copies for hCFTR/fCFTR for each sample. 1 Is equal to endogenous levels of CFTR (red dashed line). Lung samples contained on average 3.0+/−0.5 copies of transgene derived hCFTR mRNA per copy of fCFTR mRNA. Trachea and nasal tissue transduction was more variable, but averaged one copy of transgene derived hCFTR/fCFTR mRNA. Results depict the mean+/−SEM for the vector treated animals.

### DETAILED DESCRIPTION

Gene therapy has been widely used in clinical trials since 1990s with many successful cases reported using viral or non-viral vectors to deliver therapeutic genes. rAAV is the most widely used one proven of high safety profile, broad tissue/organ tropism and persistence transgene expression. AAV is a small single stranded DNA virus of an inherently small 4.679 kb genome, thus the application of rAAV for gene therapy is restricted to delivering relative small transgenes. Although AAV capsid can house a rAAV genome slightly larger than its original size, 4.95 kb appears to be the maximal size for efficient transgene expression. Since a 300-bp sequence of an AAV essential cis element (terminal repeats at both termini) Is included in a rAAV vector, the actual insertion of an exogenous gene expression cassette cannot exceed 4.6 kb. This is a challenge for delivering effective expression of a large gene whose size approaches to this limit.

One typical example is to deliver the CFTR gene (cystic fibrosis transmembrane conductance regulator) for cystic fibrosis (CF) gene therapy using rAAV vector. The coding sequence for CFTR gene is as large as 4.443 kb. To construct a CFTR expressing AAV vector, with the necessity of minimal 5′ and 3′ UTR and the cloning sites, there is a room of less than 200 bp to incorporate promoter and polyadenylation signal to direct the transcription of full-length CFTR cDNA.

Recently, a CFTR knockout ferret model was established that spontaneously develops a lung phenotype that mirrors key features of human CF disease, including spontaneous bacterial infection of the lung, defective secretion from submucosal glands, diabetes, and gastrointestinal disease (Sun et al., 2008; Sun et al., 2010; Oliver et al., 2012; Sun et al., 2014; Yan et al., 2013. It has been demonstrated that the airways of newborn ferrets can be efficiently transduced by rAAV1 in the presence of proteasome inhibitors (Yan et al., 2013). Thus, preclinical studies in the CF ferret model can be initiated as soon as a rAAV vector that effectively expresses CFTR in airway epithelium is generated. rAAV inherently small 4.679 kb genome necessitates the use of a short but robust transcription regulatory element to effectively express a large transgene whose size approaches to the package limit. cassette was generated that efficiently expresses the ferret CFTR (fCFTR) gene.

The first-generation rAAV-CFTR vector (AV2.tgCF), relied on the cryptic promoter activity of the AAV2 ITR, inefficiently expressed CFTR in clinical trials. To overcome this problem, another rAAV vector, AV2.tg83-CFTR, which uses an 83-bp synthetic promoter (tg83) was used to improve expression. Although this vector produced a 3-fold higher in cAMP-mediated Cl⁻ currents in CF HAE ALI cultures than AV2.tgCF, this level of expression remains suboptimal for application in CF gene therapy. So, there is an immediate need for a strong short promoter to direct the CFTR expression in the AAV vector for CF gene therapy. Similarly, to express the 4.3 kb B-domain deleted Factor-VIII in muscle and/or liver for hemophilia gene therapy using rAAV, short promoter effective in muscle and liver is also needed.

Another example is to deliver the CRISPR/Cas9 system for gene editing. The recent development of CRISPR/Cas9 gene editing technique promotes a new human gene therapy strategy by correcting a defect gene at pre-chosen sites without altering the endogenous regulation of gene of interest. This system consists of two key components: Cas9 protein and sgRNA, as well as a correction template when needed. rAAV can be used to deliver these elements in vivo to various target organs, but the co-delivery of Cas9 protein and the a chimeric sgRNA in the same cell is required while the dual-AAV vector delivery system is low efficient. Because the size of the expression cassette for Streptococcus pyogenes (SpCas9) and the transcription cassette sgRNA together exceeds 4.2 kb, to use a single rAAV vector to deliver the efficient expression SpCas9 protein, it necessitates the use of small but robust promoter/enhance sequence to direct the SpCas9 expression, thus, ubiquitous and/or tissue-specific enhancers are desired. Although Staphylococcus aureus Cas9 (SaCas9), which is about 1.0 kb smaller in size, fits together with its sgRNA and relevant expression cassettes within a single AAV vector, using short synthetic promoter allows for the additional incorporation of the gene correction template for an all-in-one rAAV vector in the application of gene editing-based gene therapy.

As described below, short (less than 0.2 kb) synthetic enhancer/promoters provide a solution to solve the current problem of rAAV vector in delivering a large transgene cassette. This disclosure, in one embodiment, relates to the use of a 183-bp F5tg83 synthetic enhancer/promoter to rAAV vectors to deliver effective CFTR expression in lung airway tissue for CF gene therapy. This disclosure, in one embodiment, also provides an effective approach to screen and identify tissue-specific or ubiquitous synthetic promoter/enhancer combinations.

Since enhancer activity differs by cell lines and state of cell differentiation, as well as is influenced by the AAV ITRs and by the sequence of gene of interest, the screening was conducted in a step-wise fashion, e.g., in plasmids, proviral vectors, and rAAV vectors.

In one embodiment, the screening system includes a defined 83-mer synthetic core promoter (tg83p) and a set of random 100-mer synthetic sequence of potent enhancer activity. The screening approach can be used to screen the 100-mer synthetic sequences for their enhancer activity to enhance promoter transcription, e.g., the 83 bp tg83p promoter transcription, in different organ/tissue for different gene of interests, in a similar in a step-wise fashion: such as to direct the Factor Villi expression in muscle or liver, as well as to direct the Cas9 protein expression in any specific tissues or stem cells. Besides tissue-specific expression, the approach also can be used to identify an enhancer of ubiquitous effect to improve the tg83p promoter activity in a wide range of tissue/organ, through testing rAAV derived reporter gene expressions at a multi-organ level.

Specifically, a set of vectors containing the synthetic tg83 promoter linked with different synthetic sequences (about 100 bp) of potent enhancer activity, was constructed for initial screening in monolayer (non-polarized) cultures of human airway cell lines and primary ferret airway cells, which as discussed below revealed that three of these synthetic enhancers (F1, F5, and F10) significantly promoted transcription of a luciferase transgene from tg83p in the context of plasmid transfection. The next was to construct rAAV reporter vectors with pre-chosen candidates (F1-, F5-, or F5-tg83p enhancer/prompter combination). These vectors also incorporated a partial sequence of the gene of interest (CFTR here) that can maximally fit into the rAAV genome; this approach allows for the screening of cDNA sequences that will ultimately reside in the recombinant virus and also influences enhancer/promoter activity through unknown processes (likely secondary structure of the DNA). Analysis in polarized cultures of human and ferret airway epithelia at an air-liquid interface (ALI) in the context of AAV vector infection found that the combination of F5tg83 (183 bp in length) was the most efficient promoter in both ALI cultures, leading to 19.6-fold and 57.5-fold increases in reporter (firefly luciferase) expression, respectively, over the enhancer-less counterpart. The F5tg83 promoter also produced the highest level of transgene expression in the ferret airway in vivo. Finally, the F5tg83 promoter was used the rAAV-CFTR vector to direct the CFTR expression, the vector (AV.F5tg83CFTRΔR) yielded an about 17-fold increase related to the enhancer-less vector (AV.tg83CFTRR) in vector derived CFTR mRNA transcription and significantly improved Cl⁻ currents in human CF ALI cultures.

Thus, expression from rAAV vectors having a large transgene was enhanced using small synthetic enhancer/promoter combinations having from a defined 83-mer synthetic core promoter and a set of random synthetic 100-mer synthetic enhancers. In particular, several short 183 bp synthetic promoter/enhancer combinations (F5tg83, F1tg83 and F10tg83) were capable to direct strong transgene expression in human as well as non-human mammalian (such as ferret) airway cells. In one embodiment, the robust F5tg83 promoter can be used in rAAV vector to deliver the 4.4 kb cystic fibrosis transmembrane conductance regulator (CFTR) for cystic fibrosis gene therapy.

The invention will be further described by the following non-limiting examples.

### Example 1

### Materials and Methods

Production of rAAV Vectors. All rAAV vector stocks were generated in HEK293 cells by triple plasmid co-transfection using an adenovirus-free system, and purified with two rounds of CsCl ultracentrifugation as reported in Yan et al. (2004). For all viral vectors and proviral plasmids, rAAV2 genomes were used and packaged into AAV2 or AAV1 capsid to generate rAAV2/2 and rAAV2/1 viruses, respectively. TaqMan real-time PCR was used to quantify the physical titer (DNase resistant particles, DRP) of the purified viral stocks as described in Yan et al. (2006) and Ding et al. (2006). The PCR primer/probe set used to titer luciferase vectors was: 5′-TTTTTGAAGCGAAGGTTGTGG-3′ (forward primer) (SEQ ID NO:1), 5′-CACACACAGTTCGCCTCTTTG-3′ (reverse primer) (SEQ ID NO:2) and 5′-FAM-ATCTGGATACCGGGAAAACGCTGGGCGTTAAT-TAMRA-3′ probe) (SEQ ID NO:3); the primer/probe set used for ferret CFTR vectors was 5′-GACGATGTTGAAAGCATACCAC-3′ (forward primer) (SEQ ID NO:4), 5′-CACAACCAAAGAAATAGCCACC-3′ (reverse primer) (SEQ ID NO:5) and 5′-FAM-AGTGACAACATGGAACACATACCTCCG-TAMRA-3′ (probe) (SEQ ID NO:6). All primers and probes were synthesized by IDT (Coralville, IA). The PCR reaction was performed and analyzed using a Bio-Rad My IQ™ Real-time PCR detection system and software.

Analysis of Integrity of Viral Genomes. Viral DNA was extracted from 10⁹ DRP of AAV-CFTR vectors and resolved in 0.9% alkaline denatured agarose gel at 20 volts overnight in 50 mM NaOH/1 mM EDTA buffer. Following transfer to a Nylon membrane, Southern blotting was performed with a ³²P-labeled CFTR probe to visualize the viral DNA. For examination of 5′ end genome deletions in the oversized rAAV vectors, 3.33×10⁸ DRP of each virus (quantitated by TaqMan PCR with probe/primer set against fCFTR cDNA) was loaded into a slot blotting Nylon membrane. The blots were first hybridized to a set of three ³²P-labeled oligonucleotide probes against the minus strand of the rAAV genome: at the 5′ sequence of the tg83 promoter taccctcgagaacggtgacgtg (SEQ ID NO:7); the center of ferret CFTR cDNA: ggagatgcgcctgtctcctggaatg (SEQ ID NO:8); and the 3′ sequence of the synthetic polyA: gcatcgatcagagtgtgttggttttttgtgtg (SEQ ID NO:9). After exposure to X-film, the membranes were stripped of probe and hybridized again to another set of three ³²P-labeled oligonucleotide probes complimentary to the positive strand. NIH ImageJ software was used to quantify the signal intensity of hybridization to determine the corresponding number of genomes detected by each probe with serial dilutions of the proviral plasmid as standards.

Cell Culture and Conditions for Transfections and Infections. Human airway cell lines A549 and IB3, as well as HEK 293 cells, were cultured as monolayers in Dulbecco's modified Eagle medium (DMEM), supplemented with 10% fetal bovine serum and penicillin-streptomycin, and maintained in a 37° C. incubator at 5% CO₂. Primary ferret airway cells were isolated and cultured as non-polarized monolayer or at an ALI to generate polarized epithelia as described in Liu et al. (2007). Polarized primary HAE were generated from lung transplant airway tissue as described in Karp et al. (2002) by the Cells and Tissue Core of The Center for Gene Therapy at the University of Iowa. Polarization of cells of the CuFi8 line, a conditionally transformed cell line that was generated from ΔF508/ΔF508 CF airway cells (Zabner et al., 2003), were polarized at an ALI using conditions similar to those used for primary HAE (Yan et al., 2013). Ferret and human airway epithelia were grown on 12 mm Millicell membrane inserts (Millipore) and differentiated with USG medium of 2% Ultroser G supplement (Pall BioSepra, SA, France) at an ALI prior to use. Cell lines and primary monolayer cultures of airway cells were transfected with plasmids using lipofectamine and 1.0 μg of plasmid. For rAAV infections of A549 cells, polarized human or ferret airway epithelial cells, vectors were typically left in the culture medium for 24 hours (A549 cells) or 16 hours (polarized cells). For apical infection of the polarized HAE ALI cultures, vectors were diluted in USG medium to a final volume of 50 μL and applied to the upper chamber of the Millicell insert. For basolateral infections, vectors were directly added to the culture medium in the bottom chamber. Proteasome inhibitors were supplied in the culture medium throughout the period of infection to polarized cells, at 40 μM LLnL (N-Acetyl-L-leucine-L-leucine-L-norleucine) and 5 μM doxorubicin in the case of polarized human, and 10 μM LLnL and 2 μM doxorubicin in the case of CuFl ALI cultures and ferret ALI cultures. Epithelia were exposed to the viruses and chemicals for 16 hours and then removed. At this time, the Millicell inserts were briefly washed with a small amount USG medium and fresh USG medium was added to the bottom chamber only. Doxorubicin was from Sigma (St, Louis, MO) and LLnL was from Boston Biochem (Cambridge, MA).

rAAV Infection of Ferret Lunas. All animal experimentation was performed according to protocols approved by the Institutional Animal Care and Use Committee of the University of Iowa. In vivo infection of ferret lungs was performed by intra-tracheal injection of a 300 μl inoculum containing 2×10¹¹ DRP of rAAV2/1 and 250 μM doxorubicin. Prior to infection at 5 days of age, ferret kits were anesthetized by inhalation of a mixture of isofluorane and oxygen. At 8-day post-infection, the animals were euthanized with an overdose sodium pentobarbital intraperitoneal injection. For luciferase expression assays, the ferret trachea and lung cassette was immediately frozen in liquid nitrogen and then pulverized using a cryogenic tissue pulverizer. 1 ml of Passive Lysis Buffer (Promega, Madison, WI) was added to the pulverized tissue to extract protein. After four freeze-thaw cycles, the tissue extract was centrifuged at 15,000 rpm for 5 minutes, and the clarified tissue extract was used for luciferase assays with a luciferase assay kit from Promega.

Measurement of Expression of the Firefly Luciferase Reporter. At the indicated times post-infection or transfection, cells were lysed with luciferase cell lysis buffer and luciferase enzyme activity in cell lysates was determined using the Luciferase Assay System (Promega) in a 20/20 luminometer equipped with an automatic injector (Turner Biosystems, Sunnyvale. CA).

Measurement of Short-Circuit Currents. Transepithelial short circuit currents (Isc) were measured using an epithelial voltage clamp (Model EC-825) and a self-contained Ussing chamber system (both purchased from Warner Instruments, Inc., Hamden, CT) as described in Liu et al. (2007). Throughout the experiment the chamber was kept at 37° C., and the chamber solution was aerated. The basolateral side of the chamber was filled with buffered Ringer's solution containing 135 mM NaCl, 1.2 mM CaCl₂, 1.2 mM MgCl₂, 2.4 mM KH₂PO₄, 0.2 mM K₂HPO₄, and 5 mM Hepes, pH 7.4. The apical side of the chamber was filled with a low-chloride Ringer's solution in which 135 mM Na-gluconate was substituted for NaCl. Transepithelial voltage was clamped at zero, with current pulses applied every 5 seconds and the short-circuit current recorded using a VCC MC8 multichannel voltage/current clamp (Physiologic Instruments) with Quick DataAcq software. The following chemicals were sequentially added to the apical chamber: (1) amiloride (100 μM), to inhibit epithelial sodium conductance by ENaC; (2) 4,4′-diisothiocyanato-stilbene-2,2′-disulfonic acid (DIDS) (100 μM), to inhibit non-CFTR chloride channels; (3) the cAMP agonists forskolin (10 μM) and 3-Isobutyl-I-methylxanthine (IBMX) (100 μM) to activate CFTR chloride channels; and (4) the CFTR inhibitor GlyH-101 (N-(2-naphthalenyl)-[(3,5-dibromo-2,4-dihydroxyphenyl) methylene] glycine hydrazide) (10 μM) to block Cl⁻ secretion through CFTR. ΔIsc was calculated by taking the difference of the plateau measurement average over 45 seconds before and after each change in conditions (chemical stimulus).

Quantitative Analysis of Vector-Derived CFTR mRNA Following Transduction with rAAV. The total RNA from rAAV-Infected cells was prepared using the RNeasy Mini plus Kit (Qiagen). Since the residual ssDNA rAAV genome in the RNA sample can be an undesirable template for traditional Real Time PCR, a modified RNA-specific method for PCR of the rAAV vector⁴⁶ was used to detect the vector-derived ferret CFTR mRNA. In brief, the 1^{st}-strand cDNA synthesis was primed with an adapter (lower case)-inked, vector-specific primer that targets the synthetic polyadenylation signal sequence (upper cases). The sequence of this primer is 5′-gcacgagggcgacugucaUGAUCGAUGCAUCUGAGCUCUUUAUUA-3′ (SEQ ID NO:10), in which all dTs are replaced with dU. After RNase H digestion was carried out to eliminate the RNA templates, a ferret CFTR-specific primer (5′-TGCAGATGAGGTTGGACTCA-3′; SEQ ID NO:11) was used for synthesis of the 2^{nd} strand. In order to avoid false amplification from cDNA produced from the single-stranded viral DNA, all of the dU components in the 1^{st}- and 2^{nd}-strand cDNA products, as well as the excess adapter primers, were degraded by applying uracyl-N-glycosylase (UNG). Thus, a 2^{nd}-strand cDNA product linked to the complementary sequence of the adapter derived exclusively from rAAV transcripts was produced. The primer set for TaqMan PCR contained the ferret CFTR sequence 5′-CAAGTCTCGCTCTCAAATTGC-3′ (SEQ ID NO:12), and the adapter sequence 5′-GCACGAGGGCGACTGTCA-3′ (SEQ ID NO:13). The TaqMan probe used was 5′-FAM-ACCTCTTCTTCCGTCTCCTCCTTCA-TAMRA-3′ (SEQ ID NO:14).

### Results

Synthetic Oligonucleotide Enhancers that Increase Tg83 Promoter-Driven Transcription in Airway Cells.

A previous unbiased screen evaluating short synthetic enhancers from a library containing 52,429 unique sequence identified enhancer elements capable of activating transcription from the 128 bp minimal cytomegalovirus (CMV) IE promoter (−53 to +75) in HeLa cells (Schlabach et al., 2010). This library comprised all possible 10-mer DNA sequences, printed on microarrays as 10 tandem repeats (for a total length of 100 bases each). The best-performing 100-mer oligonucleotides enhanced the transcription of this 128 bp CMV IE minimal promoter to 75%-137% of that induced by the 600 bp wild type CMV IE promoter (Schlabach et al., 2010). In previous studies, a 83 bp synthetic promoter sequence (tg83) was used to express the full-length CFTR gene from a rAAV vector (AV2.tg83-CFTR), and it was found to produce higher transgene expression in CF HAE cultures than the cryptic promoter of the AAV2 ITR (Zhang et al., 2009). The tg83 promoter consists of an ATF-1/CREB site and an Sp1-binding site from the promoter of the Na,K-ATPase α1 subunit, and the TATA box and transcription start site from the CMV IE promoter. It was hypothesized that combining the tg83 promoter with a synthetic enhancer identified through this library screen would produce transcriptional units of greater efficiency in polarized human and/or ferret airway epithelia in vitro and in vivo. To test this possibility, the top eight enhancer sequences identified by Schlabach et al. (F1, F4, F5, F10, C9, D3, CREB6 and CREB8; Schlabach et al., 2010) were evaluated for their ability to enhance tg83 transcription in human and ferret airway epithelium.

F1
(SEQ ID NO: 15)
AGTCAGGGCAAGTCAGTGGCAAGTCAGGGCAGTCAGGGCAGTCAGGGCAA
GTCAGGGCAAGTCAGGGCAAGTCAGGGCAAGTCAGGGCAAGTCAGGGCA
F10
(SEQ ID NO: 16)
gaattgacgcatatattgacgcatattgacgcaaattgacgcaaatgaca
gcaagattgacgcaaattgagcgcaaattgacgcaaattaattgacgcat
F4
(SEQ ID NO: 17)
CTGATGCAATCTGATGCAATCTGATGCAATCTGATGCAATCTGATGCAAT
CTGAGCAATCTGATGCAATCTGATGCAATATGATGAATGTGATGCAAT
F5
(SEQ ID NO: 18)
TGGTGAGCGTCTGGGCATGTCTGGGCATGTCTGGGCATGTCTGGGCATGT
CGGGCATTCTGGGCGTCTGGGCATGTCTGGGCATGTCTGGGCA
C3
(SEQ ID NO: 19)
GCTGATGCAATCTGATGCAATCTGATGCAATCTGATGCAATCTGATGCAA
TCTGAGCAATCTGATGCAATCTGATGCAATATGATGAATGTGATGCAATT
D9
(SEQ ID NO: 20)
GCTGATGCAATCTGATGCAATCTGATGCAATCTGATGCAATCTGATGCAA
TCTGAGCAATCTGATGCAATCTGATGCAATATGATGAATGTGATGCAATT
CREB6
(SEQ ID NO: 21)
ATTGACGCGGATTGACGCGGATTGACGCGGATTGACGCGGATTGACGCGG
ATTGACGCGG
CREB8
(SEQ ID NO: 22)
ATTGACGCGGATTGACGCGGATTGACGCGGATTGACGCGGATTGACGCGGA
TTGACGCGGATTGACGCGGATTGACGCG

### Enhancer/Promoter Combinations:

tg83
(SEQ ID NO: 23)
ctcgagaacggtgacgtgcacgcgtgggcggagccatcacgcaggttgct
atataagcagagctcgtttagtgaaccgtcaga
F1tg83
(SEQ ID NO: 24)
AGTCAGGGCAAGTCAGTGGCAAGTCAGGGCAGTCAGGGCAGTCAGGGCAA
GTCAGGGCAAGTCAGGGCAAGTCAGGGCAAGTCAGGGCAAGTCAGGGCAc
tcgagaacggtgacgtgcacgcgtgggcggagccatcacgcaggttgcta
tataagcagagctcgtttagtgaaccgtcaga
F10tg83
(SEQ ID NO: 25)
GAATTGACGCATATATTGACGCATATTGACGCAAATTGACGCAAATGACA
GCAAGATTGACGCAAATTGAGCGCAAATTGACGCAAATTAATTGACctcg
agaacggtgacgtgcacgcgtgggcggagccatcacgcaggttgctatat
aagcagagctcgtttagtgaaccgtcaga
F4tg83
(SEQ ID NO: 26)
CTGATGCAATCTGATGCAATCTGATGCAATCTGATGCAATCTGATGCAAT
CTGAGCAATCTGATGCAATCTGATGCAATATGATGAATGTGATGCAATct
cgagaacggtgacgtgcacgcgtgggcggagccatcacgcaggttgctat
ataagcagagctcgtttagtgaaccgtcaga
F5tg83
(SEQ ID NO: 27)
GTGGTGAGCGTCTGGGCATGTCTGGGCATGTCTGGGCATGTCTGGGCATG
TCGGGCATTCTGGGCGTCTGGGCATGTCTGGGCATGTCTGGGCATctcga
gaacggtgacgtgcacgcgtgggcggagccatcacgcaggttgctatata
agcagagctcgtttagtgaaccgtcaga
C3tg83
(SEQ ID NO: 28)
GCTGATGCAATCTGATGCAATCTGATGCAATCTGATGCAATCTGATGCAA
TCTGAGCAATCTGATGCAATCTGATGCAATATGATGAATGTGATGCAATT
ctcgagaacggtgacgtgcacgcgtgggcggagccatcacgcaggttgct
atataagcagagctcgtttagtgaaccgtcaga
D9tg83
(SEQ ID NO: 29)
GCTGATGCAATCTGATGCAATCTGATGCAATCTGATGCAATCTGATGCAA
TCTGAGCAATCTGATGCAATCTGATGCAATATGATGAATGTGATGCAATT
ctcgagaacggtgacgtgcacgcgtgggcggagccatcacgcaggttgct
atataagcagagctcgtttagtgaaccgtcaga
CREB6tg83
(SEQ ID NO: 30)
ATTGACGCGGATTGACGCGGATTGACGCGGATTGACGCGGATTGACGCGG
ATTGACGCGGctcgagaacggtgacgtgcacgcgtgggcggagccatcac
gcaggttgctatataagcagagctcgtttagtgaaccgtcaga

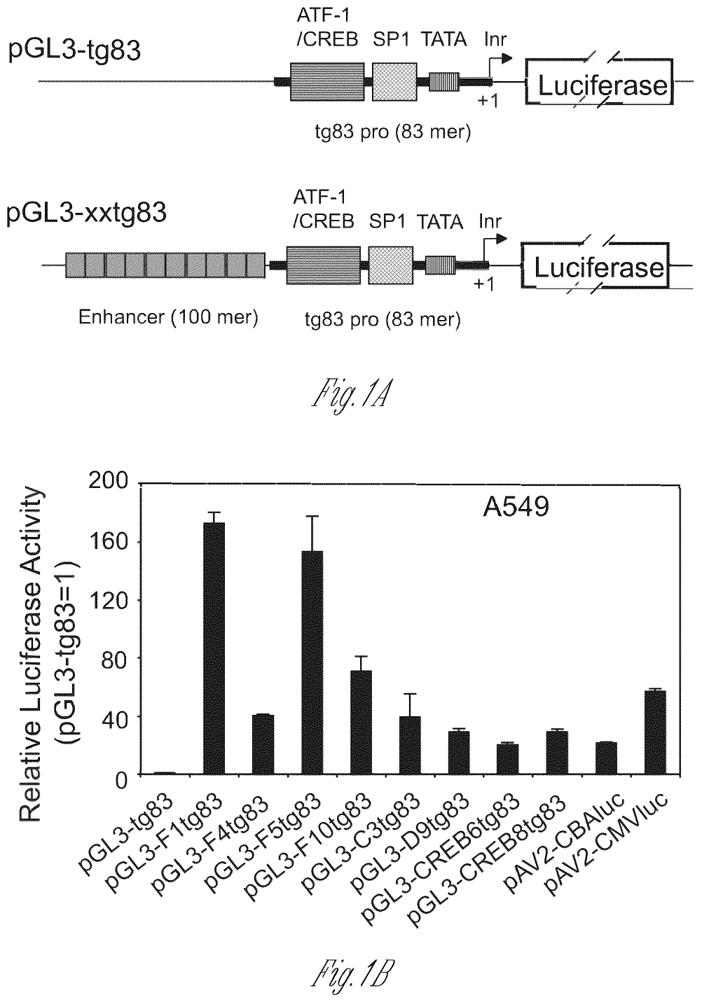

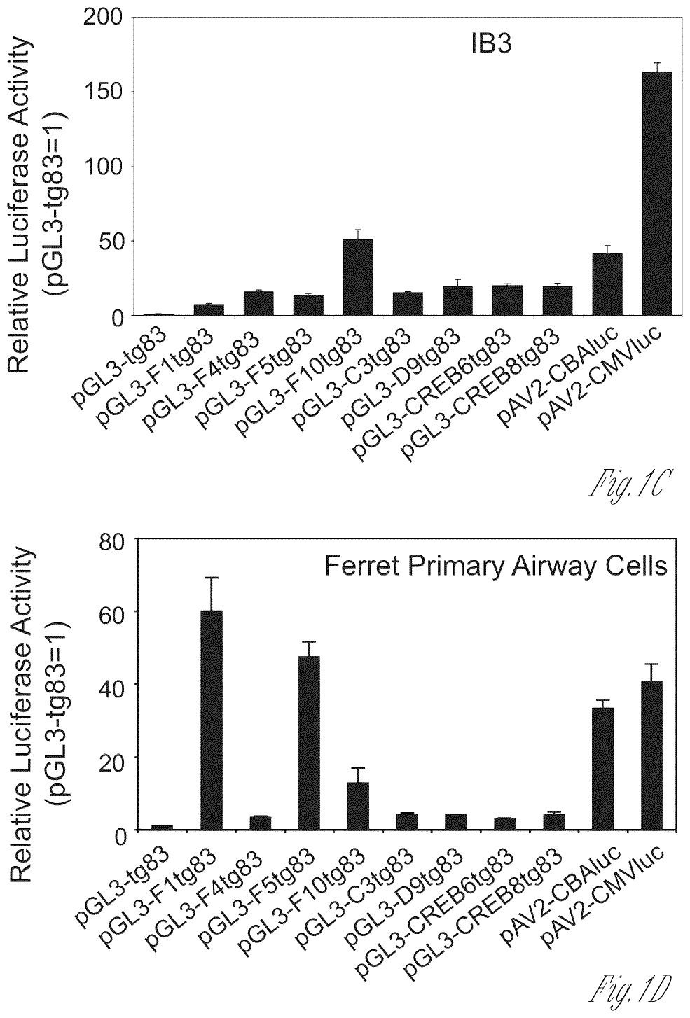

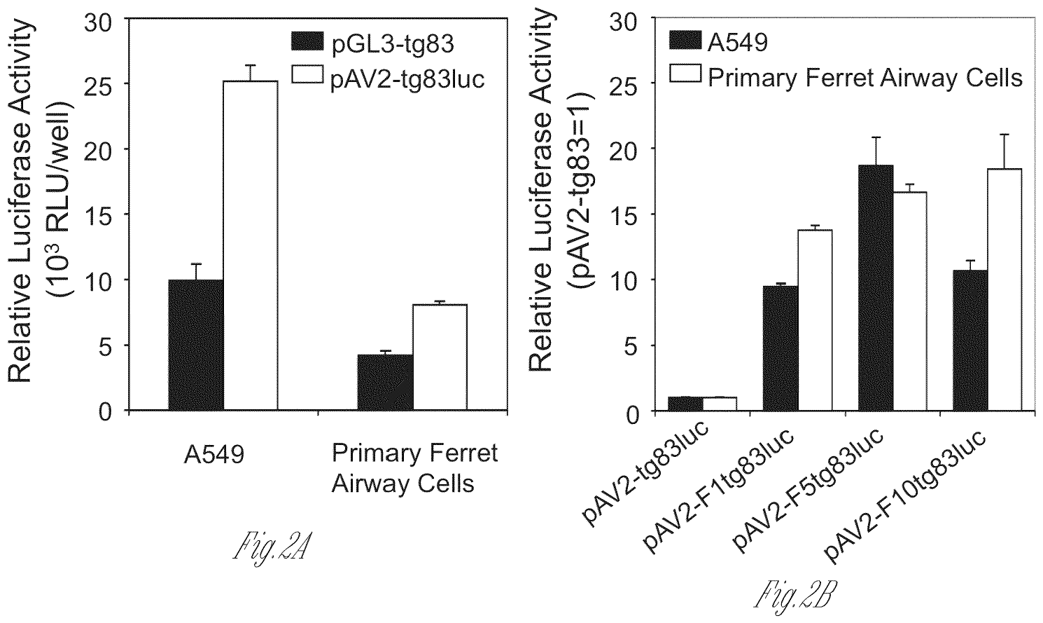

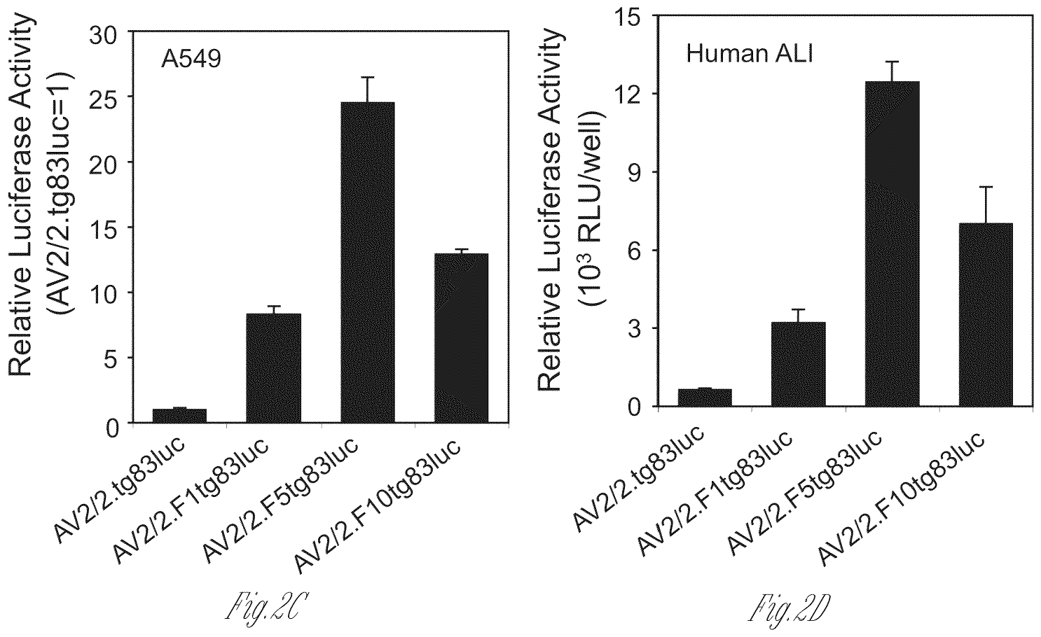

The tg83 promoter was cloned into the promoter-less luciferase reporter plasmid pGL3-Basic Vector (Promega) to generate pGL3-tg83. Next a series of luciferase reporter expression plasmids were constructed, in which one of the eight 100-mer enhancers was placed in front of the tg83 promoter of pGL3-tg83 (FIG. 1A). Comparison of reporter expression from pGL3-tg83 and its enhancer-containing derivatives was conducted in monolayer (non-polarized) cultures of two human airway cell lines (A549 and IB3) and primary ferret airway cells (FIGS. 1B-1D). Two additional luciferase expression plasmids, pAV2-CMV-luc (contains the wild type, 800 bp CMV IE enhancer-promoter) and pAV2-CBA-luc (contains the CMV IE enhancer-chicken β-actin promoter, i.e., the CBA promoter) were included as controls for high-level promoter activity. Assessment of luciferase expression following plasmid transfection demonstrated that all of the enhancers tested increased tg83-driven luciferase expression, and that their efficiencies varied by cell line: in the human A459 cell and the primary ferret airway cell cultures, F1tg83 and F5tg83 exceeded the activity of the CBA and CMV promoters; and in the human IB3 cell cultures, F10tg83 was most effective but drove far less expression than the CMV promoter (FIGS. 1B-1D).

### The F5 Element Most Efficiently Enhances Tg83-Driven Transcription in Polarized Human and Ferret Airway Epithelia In Vitro, as Well as in the Ferret Airway In Vivo.

Since the F1, F5 and F10 enhancers were the most effective in activating tg83-driven transcription in airway-cell monolayer cultures, the abilities of these elements to promote transcription in the context of rAAV vector genomes was evaluated. Four rAAV proviral vectors harboring a luciferase expression cassette were constructed, with expression driven by tg83 (enhancer-less), F1tg83, F5tg83 or F10tg83. The pAV2-tg83-fCFTR proviral plasmid was used as the template vector for cloning, its promoter and the 5′ portion of the fCFTR coding region were replaced with the 2.1 or 2.2 kb luciferase expression cassette. The genome size was 4.75 kb in the case of rAV2.tg83luc, and 4.85 kb for the enhancer-containing vectors. This design was used for two reasons. First, retaining as much of the fCFTR sequence as possible ensured that the vector genome size would be similar to those of the rAAV-CFTR expression vectors that would ultimately be generated. Second, retaining regions of the fCFTR cDNA maximized the potential influences of the ferret CFTR sequence on enhancer function.

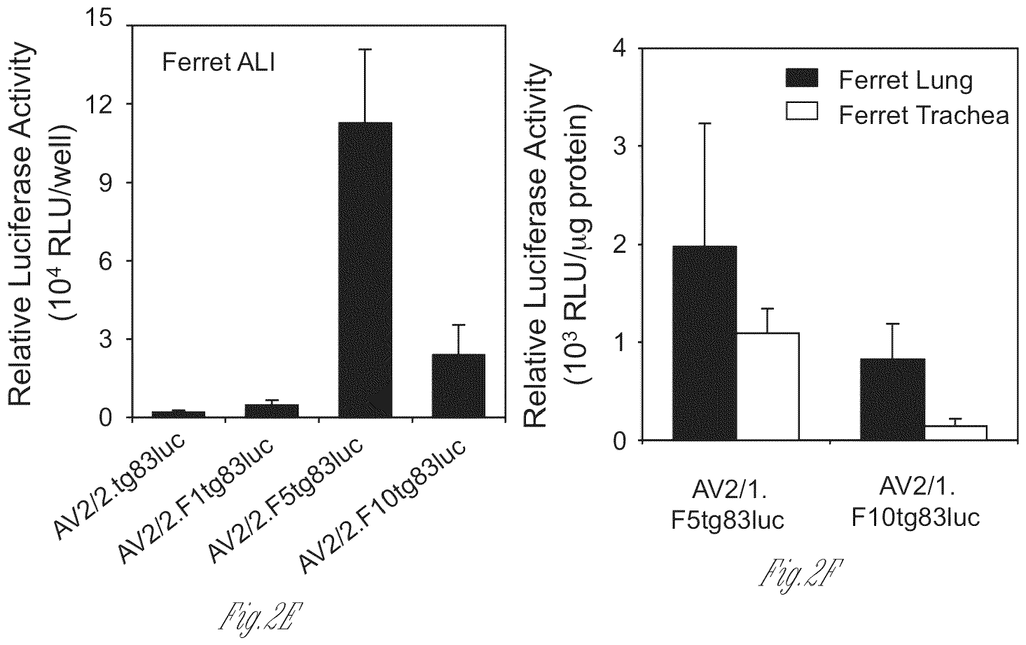

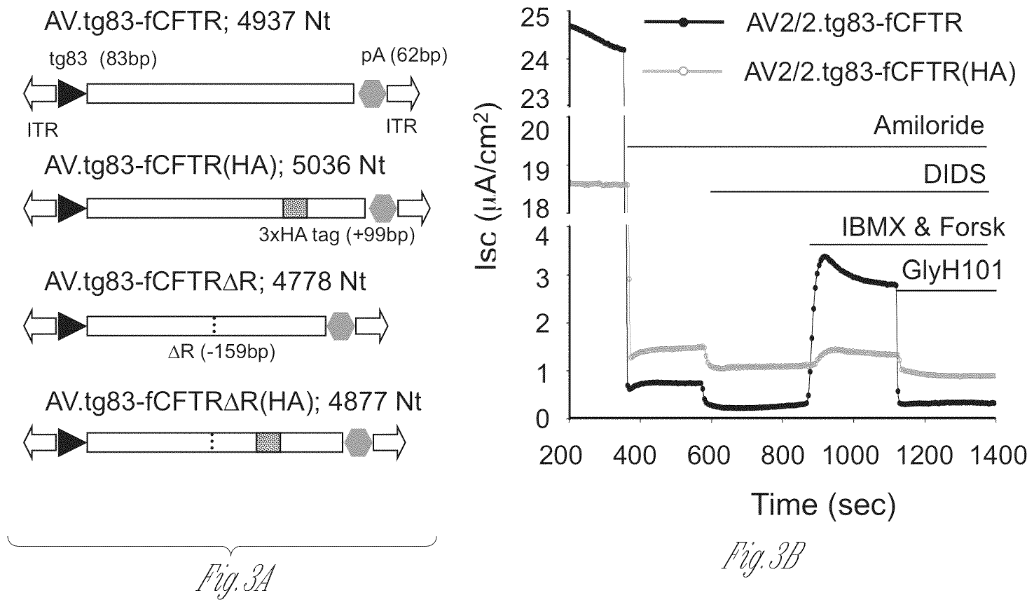

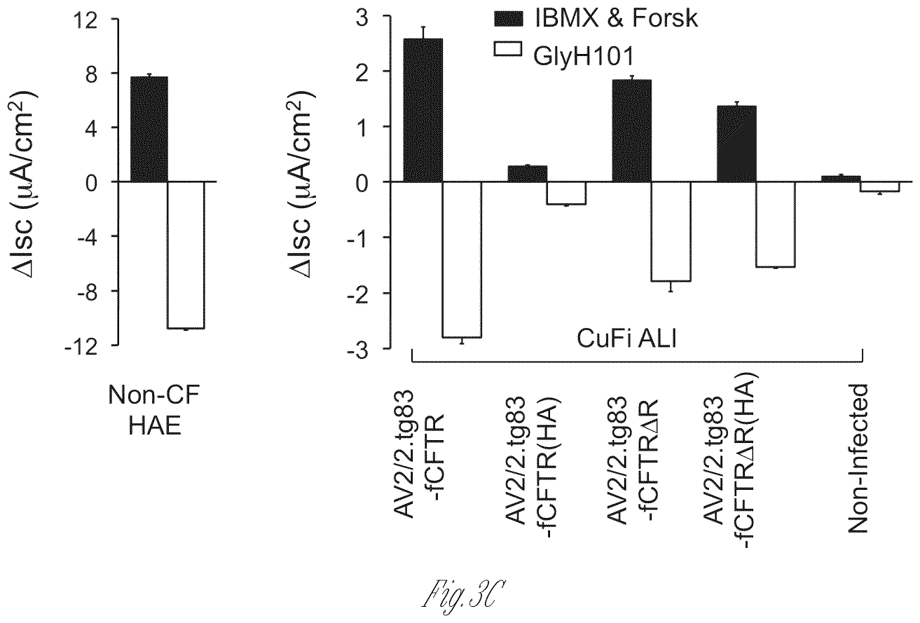

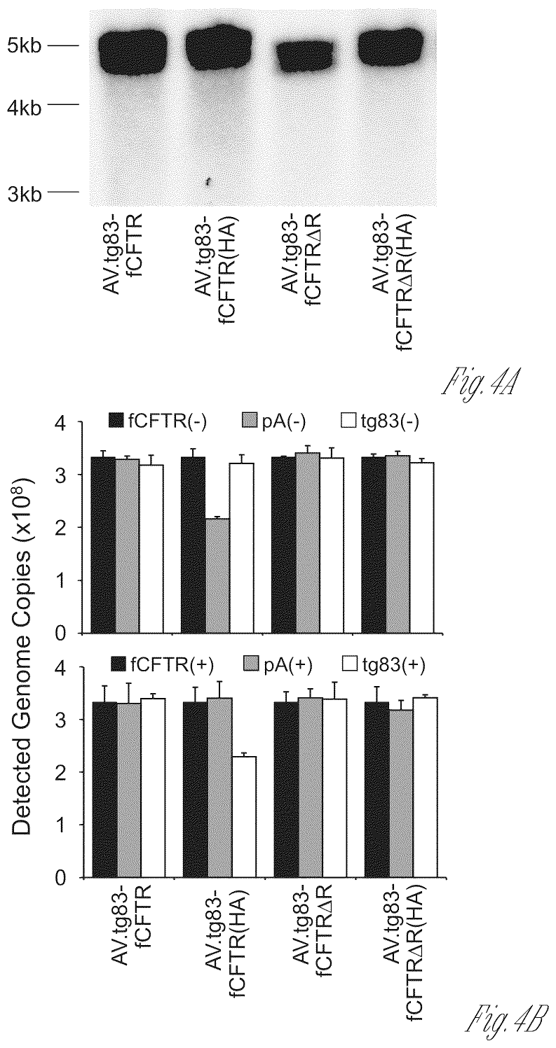

As a first step in investigating whether AAV ITRs and the portion of fCFTR transgene sequence to be tested (i.e., 3′ half of the fCFTR cDNA) influence transcription from the tg83 promoter, reporter expression from pGL3-tg83 and pAV2-tg83luc plasmids was compared following transfection into monolayer cultures of A549 and primary ferret airway cells. pAV2-tg83luc plasmid was found to be 2.5-fold (in A549) and 2-fold (in primary ferret airway cells) more transcriptionally active than the pGL3-based plasmids (FIG. 2A), suggesting that inclusion of the AAV ITR and/or the fCFTR stuffer sequences had an overall positive effects on activity of the tg83 promoter. Then reporter-gene expression for pAV2-F1tg83luc, pAV2-F5tg83luc, pAV2-F10tg83luc, and pAV-tg83luc plasmids was compared. As expected, the F1, F5 and F10 enhancers significantly improved transcription from the tg83 promoter (about 10- to 19-fold) in both cell types (FIG. 2B). However, the effectiveness of nearly all enhancers was significantly reduced (about 3- to 18-fold) within the rAAV proviral plasmids when compared to pGL3-tg83 plasmids lacking ITRs and the CFTR sequence (FIG. 2B solid bars vs. FIG. 1B; FIG. 2B open bars vs FIG. 1D). This suggests that the sequences from the AAV ITR and/or portions of the ferret CFTR cDNA have an overall negative impact on enhancer function. However, this effect on the synthetic enhancers differed between the A549- and ferret primary airway-cell monolayers. In A549 cells, the F1 enhancer was most significantly influenced, with its activity in the rAAV proviral plasmid decreased by 18.1-fold, whereas those of the F5 and F10 enhancers decreased by only 8.2-fold and 3.8-fold, respectively. In primary ferret airway cells, the F1 and F5 enhancers had 4.4-fold and 2.8-fold decreased activity, respectively, in the context of the proviral plasmid, whereas the function of F10 was slightly enhanced (about 40%).

Next expression from the various enhancer elements was evaluated in the context of rAAV2/2 vectors. In A549 cells, similar increases in expressions from the enhancer/tg83 promoter combination were observed following the transfection with the proviral plasmid and infection with the corresponding rAAV vector (FIG. 2B solid bars vs. FIG. 2C). Primary human and ferret airway epithelial ALI culture were then infected with equal titers of each rAAV vector, and transgene expression was assessed at 2 days post-infection. These experiments demonstrated that F5tg83 Is the most efficient promoter in both human and ferret ALI cultures (FIGS. 2D and 2E), leading to 19.6-fold and 57.5-fold increases, respectively, in tg83-driven transcription over that driven by the enhancer-less control (FIGS. 2D and 2E). Notably, the differentiated state of ferret airway epithelial cells appeared to dramatically influence expression from the various enhancer/tg83 promoter combinations in the context of rAAV transduction; the F5 enhancer more effectively enhanced tg83 expression in the polarized epithelium (FIG. 2E; 57.5-fold) than in undifferentiated monolayers (FIG. 2B; 16.6-fold); the F1 enhancer only marginally increased activity of the tg83 promoter in polarized cells, but increased transgene expression 13.8-fold in monolayer cells.

Lastly, the in vivo activities of the F5tg83 and F10tg83 promoters were compared in the airways of newborn ferrets, using intratracheal injection of two rAAV1 capsid pseudotyped vectors (AV2/1.F5tg83luc and AV2/1.F10tg83luc; equal particle titers injected). This capsid serotype had previously been shown to be effective at transducing the ferret airways in the presence of a proteasome inhibitor (Yan et al., 2013). Luciferase activity was measured in extracts prepared from tracheal and lung tissue at 8-day post-infection, and F5tg83 was found to be more effective than F10tg3 In transducing both ferret lung and trachea (FIG. 2F). These findings were consistent with those for polarized ferret airway epithelial ALI cultures (FIG. 2E).

A Narrow Limit for rAAV Genome Size Significantly Influences Functionality of rAAV-CFTR Vectors while not Altering Packaging Efficiency.

The size of the expected AV.tg83fCFTR genome if fully packaged is 4.937 kb (FIG. 3A). Incorporation of the F5 enhancer would increase this to 5.040 kb. Although it is well accepted that AAV can encapsidate a rAAV genome slightly longer than its natural size (4.679 kb), gradually increasing the size of a rAAV vector from 4.675 kb to 4.883 kb and 5.083 kb results in 25% and 75%, respectively, decreases in transduction (Dong et al., 1996). Furthermore, single-molecule sequencing (SMS) of the two rAAV termini following packaging of a 5.8 kb proviral genome revealed that the 5′ ITR was unstable and had incurred deletions (Kapranov et al., 2012). Given that the limits for functional genome packaging in the context of rAAV-CFTR vectors have yet to be defined, it was uncertain whether a 5.04 kb AV.F5tg83fCFTR genome would be compromised with respect to genome stability and function.

This question was addressed by constructing a 5.038 kb AV2.tg83-fCFTR(HA) vector in which the CFTR expression cassette was expanded by the addition of a 3×HA epitope tag (99 nucleotides) in the region encoding the fourth extracellular loop (ECL4) of ferret CFTR (previous studies had revealed that this insertion has no impact on chloride-channel function (Glozman et al., 2009; Fisher et al., 2012)). This vector allowed us to interrogate how size of the genome influences CFTR functionality in the absence of changes to transcription of the transgene. Two rAAV2 vectors were produced (AV2/2.tg83-fCFTR and AV2/2.tg83-fCFTR-HA; FIG. 3A), and their ability to generate CFTR-mediated chloride currents was evaluated in polarized CF HAE. Vector yields for the two viruses were nearly equivalent (AV2/2.tg83-fCFTR about 5×10⁹ DRP/μL and AV2/2.tg83-fCFTR(HA) about 3×10⁹ DRP/μL). Polarized CF HAE were cultured at an ALI and infected at the relatively high multiplicity of infection (MOI) of about 10⁵ DRP/cell (10¹¹ DRP of each rAAV2 vector per insert). At 10 days following infection, the level of CFTR expression was determined by measuring short circuit current (Isc), as described in Zhang et al. (2004) and Fisher et al. (2011). FIG. 3B shows a typical Isc trace following infection of CF HAE with AV2/2.tg83-fCFTR or AV2/2.tg83-fCFTR-HA. Amiloride and DIDS were first applied to block non-CFTR chloride channels and ENaC-mediated sodium currents, and then cAMP agonists (IBMX and forskolin) were used to induce CFTR activity. The changes in Isc following the addition of IBMX and forskolin (ΔIsc_{IMBX & Forsk}) and the subsequent addition of the CFTR inhibitor GlyH101 (ΔIsc_{glyH}) were used to evaluate the function of CFTR. These results clearly demonstrated that functional complementation of CFTR activity in CF HAE is greater following infection with AV2/2.tg83-fCFTR than with AV2/2.tg83-fCFTR-HA (FIG. 3B). The mean ΔIsc_{IBMX & Forsk} and ΔIsc_{glyH} values from these experiments are summarized in FIG. 3C. CFTR-mediated cAMP-inducible Cl⁻ currents produced by AV2/2.tg83-fCFTR(HA) were only 3.6% those in a non-CF HAE ALI cultures, but still above background levels (p<0.01). By contrast, infection with AV2/2.tg83-fCFTR produced 10-fold greater cAMP-inducible CF currents than AV2/2.tg83-fCFTR(HA) and achieved about 30% CFTR activity of non-CF HAE ALI cultures. These results demonstrate that the cutoff for retaining CFTR function is very narrow when producing oversized rAAV genomes, and that vector functionality does not depend only on the efficiency of packaging DRPs. Furthermore, these studies suggest that incorporation of the 100 nucleotide F5 enhancer into AV2/2.tg83-fCFTR, with a total genome size of 5.04 kb, may have a significant negative impact on function of the genome.

Effective Packaging of a Functional Ferret CFTR Mini Gene into rAAV

Next, the possibility of using a shortened ferret CFTR minigene was explored, to further reduce the genome size of a rAAV-CFTR vector, and to allow for incorporation of the F5 enhancer. A human CFTR minigene (CFTRΔR) with a 158 bp partial deletion of the R-domain (encoding amino acids 708-759) has been reported to retain most of the chloride-channel activity of the full-length protein (Ostedgaard et al., 2002; Ostedgaard et al., 2011). A fCFTRΔR minigene was created by deleting the 159 bp homologous sequence encoding amino acids 708-760 at the corresponding position of the human protein, and produced two additional vectors: AV2/2.tg83-fCFTRΔR (4.778 kb) and AV2/2.tg83-fCFTRΔR(HA) (4.877 kb). This pair of vectors allowed for the examination not only the function of the fCFTR minigene in CF HAE ALI cultures, but also the impact of the HA-tag insertion in the fCFTR gene. Analysis of ΔIsc_{IBMX & Forsk} and ΔIsc_{glyH} responses for these two viruses demonstrated that both AV2/2.tg83-fCFTR and AV2/2.tg83-fCFTR(HA) produced substantial CFTR-mediated Cl⁻ currents following infection of the CF HAE ALI cultures (FIG. 3C). However, the HA-tagged form produced about 20% less Cl⁻ current than AV2/2.tg83-fCFTRΔR. This finding is consistent with rAAV vectors of 4.88 kb having only about 25% of the functional activity of vectors 4.68 kb (Dong et al., 1996). Alternatively, the HA-tag may itself influence CFTR function in the context of the R-domain deletion, although in the context of full-length CFTR this ECL4 HA-tag has no impact on Cl⁻ channel function (Glozman et al., 2009; Fisher et al., 2011). Despite the larger genome size of AV2/2.tg83-fCFTR (4.9437 kb), this vector produced about 30% more CFTR-mediated current than its shorter counterpart AV2/2.tg83-fCFTRΔR (4.778 kb) (FIG. 3C). This reduction in Cl⁻ channel activity of fCFTRΔR is similar to that reported for hCFTRΔR (Ostedgaard et al., 2002). However, given the potential for reduced functionality of larger vector genomes, the impact of the R-domain deletion on the function of the ferret CFTR protein is likely greater than that for human CFTR.

To establish the impact of genome length on packaging of the rAAV vectors tested, the integrity of the viral genome was examined, using alkaline-denatured agarose gel electrophoresis followed by Southern blotting (FIG. 4A). This analysis revealed that the smallest vector genome (i.e. that of AV2.tg83-fCFTRΔR. 4.778 kb) could be distinguished from the other three viruses based on its faster migration through the gel (AV2.tg83-fCFTRΔR is 99 nucleotides shorter than the AV2.tg83-fCFTRΔR(HA) vector). However, AV2.tg83-fCFTR(HA) (5.036 kb), AV2.tg83-fCFTR (4.937 kb) and AV2.tg83-fCFTRΔR(HA) (4.887 kb) could not be distinguished from one another on the basis of this analysis. Given that it should be possible to visualize differences of both 149 nucleotides (AV2.tg83-fCFTR(HA) vs. AV2.tg83-fCFTRΔR(HA)) and 99 nucleotides (AV2.tg83-fCFTR(HA) vs. AV2.tg83-fCFTRΔR(HA), and AV2.tg83-fCFTR(HA) vs. AV2.tg83-fCFTR), these findings were interpreted as support for the notion that viral genomes larger than that of AV2.tg83-fCFTRΔR(HA) (4.887 kb) tend to incur deletions that compromise CFTR transgene expression.

The notion that deletion occurs in the context of longer genomes was further supported by the hybridization of viral genomes with two sets of plus and minus strand oligonucleotide probes at the center of CFTR cDNA, the tg83 promoter, and synthetic poly-A sequences (FIG. 4B). Results from this analysis demonstrated viral DNA from the largest AV2.tg83-fCFTR(HA) vector incurred deletions at both the 5′ ends of positive and minus strand genomes. By contrast, the 3 end of positive and minus strand AV2.tg83-fCFTR(HA) genomes remained intact, consistent with packaging of single stranded AAV genomes from the 3′ to 5 direction. The fact that the strength of hybridization at the tg83 promoter (for positive strand), and the polyA sequence (for minus strand), was lower than that of hybridization to the fCFTR cDNA suggested that these deletions were not restricted to the ITR region (i.e., that the damage extended into the CFTR expression cassette). Such deletions were not observed in the second longest vector, AV2.tg83-fCFTR, therefore, the CFTR expression cassette in this vector most likely still remains intact, although partial deletions in the ITR region likely occur as suggested from the viral DNA migration on denatured agarose gel. While deletions in the ITR regions may not directly influence expression of the CFTR transgene, they may impact the stability of viral genomes and thus indirectly influence CFTR expression. These results, together with the functional analysis, led to the conclusion that the fCFTRΔR cDNA without the HA-tag would be best suited for testing the impact of the F5 enhancer on rAAV-mediated CFTR complementation.

The synthetic F5tg83 promoter improves rAAV-Mediated CFTR Complementation.

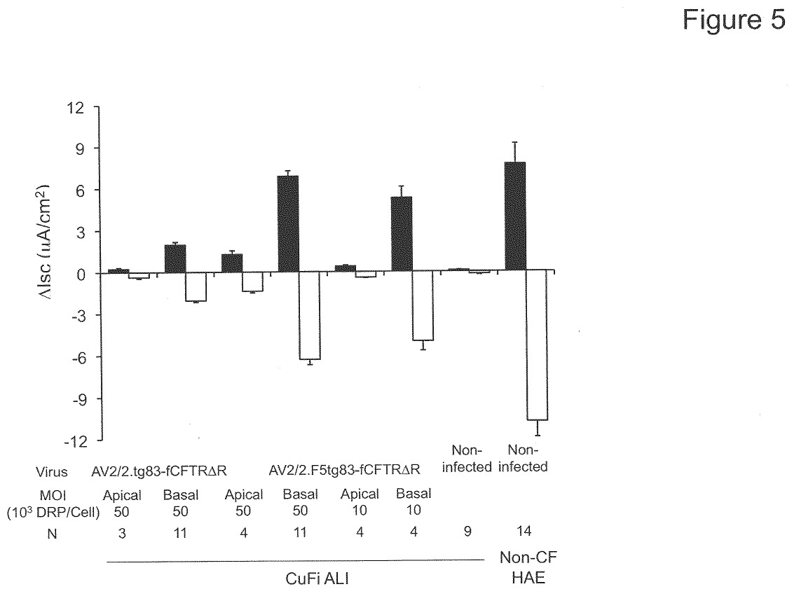

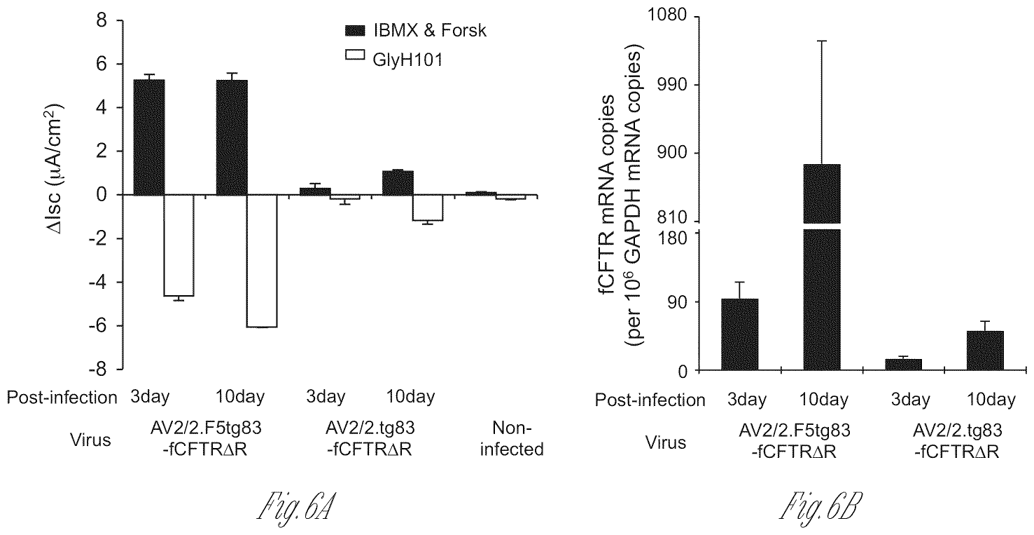

Next, the pAV2.F5tg83-fCFTRΔR proviral plasmid was generated and AV2/2.F5tg83-fCFTRΔR virus with a genome size of 4.87 kb was produced. The efficiency of this virus for complementing function of the CFTR channel following infection of polarized CF HAE was compared to that of the enhancer-less counterpart vector (AV2.tg83-fCFTRΔR). Results from this analysis demonstrated that incorporation of the F5 enhancer greatly improved the CFTR-mediated Cl⁻ currents (FIG. 5). At two weeks following basolateral infection at an MOI of 5×10⁴ DRP/cell, cAMP-Induced CFTR-mediated Cl⁻ currents were 3.5-fold greater for AV2/2.F5tg83-fCFTRΔR than for AV2/2.tg83-fCFTRΔR, and the former was 89% of those observed in non-CF primary HAE. A similar improvement in CFTR function (4.8-fold) was observed with AV2/2.F5tg83-fCFTRΔR following apical infection, but in this case the efficiency of transduction was significantly lower, as previously reported for this serotype. At the reduced MOI of 1×10⁴ DRP/cell basolaterally, AV2/2.F5tg83-fCFTRΔR produced 69% of the CFTR current generated by this vector at a 5-fold higher MOI, suggesting that complementation of CFTR function approached saturation in the latter case. Thus, when one compares the effectiveness of AV2/2.F5tg83-fCFTRΔR (1×10⁴ DRP/cell) and AV2/2.tg83-fCFTRΔR (5×10⁴ DRP/cell) vectors in the context of optimal infection (i.e., basolateral) and non-saturating conditions, incorporation of the F5 enhancer improved the vector efficacy by 13.5-fold. This level of increase in current is consistent with the increase in expression observed with the analogous luciferase expression vectors (FIG. 2D, 19.6-fold).

Given the apparent saturation of CFTR currents at the highest MOI (5×10⁴ DRP/cell) following basolateral infection with AV2/2.F5tg83fCFTRΔR, the kinetics of CFTR expression were evaluated at an intermediate MOI (2×10⁴ DRP/cell). Measurements were carried out 3 and 10 days following infection of CF HAE with AV2/2.F5tg83-fCFTRΔR and AV2/2.tg83-fCFTRΔR. Results from this analysis demonstrated that, in the context of the F5 enhancer, the onset of CFTR-mediated Cl⁻ currents was more rapid than in its absence (FIG. 6A). In fact, CFTR currents were maximal by 3 days after infection with AV2/2.F5tg83-fCFTRΔR, whereas currents increased 3.6-fold between 3 and 10 days after infection with AV2/2.tg83-fCFTRΔR. To more directly compare transcriptional activity between these vectors, the ferret CFTR mRNA was examined by real-time RNA-specific reverse transcriptase PCR (RS-PCR), a method that prevents amplification of vector-derived DNA products and was previously applied in detecting the CFTR mRNA from rAAV-infected cells and tissues (Zhang et al., 2004; Gerard et al., 2003). Analyses of the RS-PCR results, after normalization to ferret GAPDH transcripts, demonstrated 6.4-fold and 17.1-fold higher levels of fCFTR mRNA following infection with AV2.F5tg83-fCFTRΔR vs. AV2/2.tg83-fCFTRΔR, at the 3 and 10 day time points, respectively (FIG. 6B). The 10-fold increase in CFTR mRNA observed between 3 and 10 days after infection confirms that CFTR currents were saturated by 3 days post-infection. Thus, at the transcriptional level, incorporation of the F5 enhancer increased CFTR expression 17.1-fold, closely mirroring the results observed with luciferase expression vectors (FIG. 2D, 19.6-fold).

### Discussion

rAAV vectors have attracted considerable interest with respect to human gene therapy, but its inherently small genome (4.679 kb) is a significant challenge for the delivery of large genes such as CFTR. Although several laboratories have attempted to rationally design short enhancers and promoters for use in rAAV vectors, this approach has yet to yield robust expression of CFTR in the airway. In the present study, an entirely empirical approach was taken by screening synthetic enhancers for their effectiveness in the delivery of reporter gene expression in step-wise fashion—in plasmids, proviral vectors, and viral vectors. While the main goal was to develop rAAV vectors for delivering CFTR to the airway, a similar approach may prove useful for gene therapy efforts tackling the delivery of other large genes (e.g., Factor VIII and dystrophin) that necessitate the use of short promoters.

Production of an oversized rAAV genome is known to lead to random deletions at the 5′ end of the encapsidated single stranded genomes (Kapranov et al., 2012), but the functional integrity of rAAV vector genomes that approach the accepted maximum capacity of rAAV (about 5.0 kb) has not been thoroughly investigated. The current study provides, in the context of CFTR-expressing rAAV vectors, evidence that functionality of the rAAV genome begins to decline well below this 5.0 kb cut off. Evidence in support of this includes a reduction in CFTR function for 4.877 kb vs. 4.778 kb genomes (FIG. 3C) and the lack of differences in the migration of 4.877-5.038 kb single-stranded genomes when visualized on alkaline gels (FIG. 4A). Additionally, the largest CFTR vector (5.038 kb) incurred deletion in about 30% of genomes that extend beyond the 5′ ITR and into the promoter (in the case of the positive strand) or polyA region (in the case of the minus strand). This suggests that damage to the CFTR expression cassette may be responsible for the significant impairment of function of CFTR delivered by this vector. While the oligo probes used only detected deletions in 30% of genomes for this largest construct, the 90% reduction in CFTR chloride current between 4.937 vs 5.038 kb vectors suggests that a much larger percentage of genomes incur functional deletions and that ITR deletions may also impair vector performance.

The present findings seem inconsistent with previous observations of an only 4-fold change in transgene expression between rAAV vectors that are 4.7 kb and 5.2 kb in size (Wu et al., 2010). However, in this earlier study the stuffer sequence used to expand the vector was positioned between the expression cassette and the ITR, and deletions of the stuffer sequence would likely have less of an impact on the transcriptional activity of the transgene. The current study of the AAV-CFTR vector instead employed a short synthetic promoter and poly-A signal, and small deletions within these short transcriptional regulatory elements would be expected to greatly influence gene expression. Since DNase resistant particle titer is typically used to confirm effective packaging of rAAV genomes, small 5′-end deletions could have a significant impact on functionality of oversized rAAV vectors.

The empirical approach that was used to screen synthetic 100 bp enhancers for the ability to improve rAAV-mediated transgene expression in the airway yielded several important observations: 1) Enhancer activity differed by cell lines and also the state of cell differentiation. 2) Enhancer activity was influenced by the AAV ITRs and might also be influenced by the sequence of gene of interest (in this study, the 3′ half of the ferret CFTR cDNA coding region). 3) Enhancer activity in the case of rAAV infection was generally similar to that in the context of proviral vector, though with subtle differences. 4) Infection of human and ferret airways with viral vectors revealed some differences in enhancer function (most notably for AV2/2.F1tg83luc, FIGS. 2D and 2E). These results indicate that although transfection with proviral plasmid is suitable for initial screens of synthetic enhancers, performing such a screen in unpolarized primary airway cultures may not produce the same patterns of gene expression observed following AAV infection of polarized airway cultures.

Through this screen, a 100 bp synthetic enhancer (F5) was identified that significantly improves the transcriptional activity of an 83 bp synthetic promoter (tg83), in polarized cultures of both primary human and ferret airway epithelial cells, as well as in ferret lung and trachea in vivo. The ferret CFTR minigene lacking a 53 amino acid portion of the R domain (fCFTRΔR) retained about 70% of wild type fCFTR function, similar to the 80% function retained by a previously reported, analogous hCFTRΔR construct (Ostedgaard et al., 2002). Nevertheless, this was greatly compensated by the increased transgene expression from AV2.F5tg83-fCFTRΔR, as the use of the shortened minigene spares 150 bp space to allow for the incorporation of the F5 enhancer. In the context of a rAAV vector, the F5 enhancer significantly improved tg83-driven CFTR mRNA expression (17.1-fold) at 10 days post-Infection of CF HAE relative to the AV212.tg83-fCFTRΔR vector, which lacks this enhancer. This increase in CFTR mRNA expression from AV2/2.F5tg83-fCFTRΔR correlated well with the 19.6-fold improvement in CFTR-mediated currents made possible by this vector, and resulted in production of about 89% of the cAMP-mediated Cl⁻ currents observed in non-CF HAE. Of note, a ceiling on the level of functional correction that can be achieved with respect to the changes in Isc was found. In the time-course studies, maximal correction of Isc in CF HAE was achieved by 3 days post-infection, with the about 10 fold increase in CFTR mRNA by 10 days post-infection yielding no improvement in CF currents (FIG. 6). These findings provide important insight into evaluating the functionality of rAAV-CFTR vectors: dose responses of the vector are needed for accurate comparison of vector designs. The ceiling on CFTR currents could reflect self-limiting cell biology (e.g., control over the total amount of CFTR on the plasma membrane), or aberrant trafficking of CFTR to the basolateral membrane at higher levels of expression (Farmen et al., 2005).

In summary, a rAAV-CFTR vector was generated that provides high-level expression suitable for use in lung gene therapy studies in the CF ferret model. Moreover, the present findings suggest that small synthetic enhancers and promoters may be useful tools for optimizing the design of rAAV vectors for the delivery of large transgenes.

### Summary

As discussed herein, small synthetic enhancer/promoter combination, sized about or less than 200 bp, can be used in rAAV vector to deliver effective transgene expression of a large transgene expression: in this study, the gene is CFTR. As also discussed herein, empirical approach to screen a set of 100-mer synthetic enhancer elements for their ability to augment reporter expression from a short 83-bp synthetic promoter (tg83p) in lung airway tissue. Partial sequence of the gene of interest (in the study is CFTR) Is included to the reporter vector to maximize the effect to screen the best enhancer sequence. The screening was conducted in step-wise fashion—in plasmids, proviral vectors, and rAAV vectors, and in different cell/tissue levels—in monolayer (non-polarized) cultures of human airway cell lines and primary ferret airway cell, in polarized cultures of human and ferret airway epithelia at an air-liquid interface (ALI) and ferret airway in vivo. The enhancer activity differs by cell lines and state of cell differentiation, as well as is influenced by the AAV ITRs and by the sequence of gene of interest. Thus, the effects of an enhancer in the context of plasmid transfection may be different from that in the context of rAAV transduction. The in vivo effects of a particular enhancer may not be predictable from its behaviors demonstrated in culture cell lines. This, the screening needs to be conducted in different cell/tissue levels and in a step-wise fashion—in plasmids, proviral vectors, and rAAV vectors to warrant the success.

Three of synthetic enhancers (F1, F5, and F10) were found significantly increase the transcription of tg83p for luciferase transgene in the context of plasmid transfection. The F5tg83 promoter, the 183 bp combination of F5 enhancer and tg83p, was the most efficient promoter in human and ferret ALI cultures, leading to 19.6-fold and 57.5-fold increases reporter expression, respectively, over the enhancer-less counterpart. The F5tg83 promoter also produced the highest level of transgene expression in the ferret airway in vivo. When the F5tg83 promoter was used to transcribe the 4.2 kb CFTR minigene (CFTRΔR) in a rAAV vector, it yielded an about 17-fold increase in vector derived CFTR mRNA transcription and significantly improved Cl⁻ currents in human CF ALI cultures, compared to the vector using tg83p only.

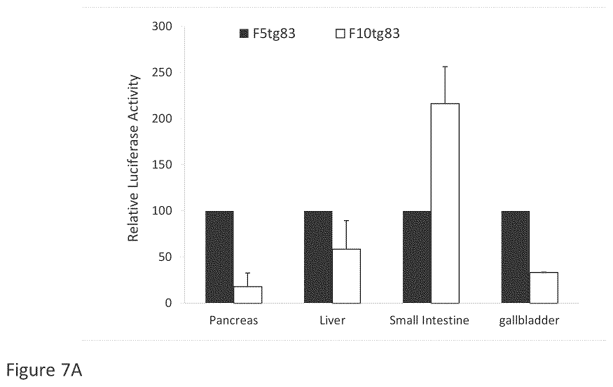

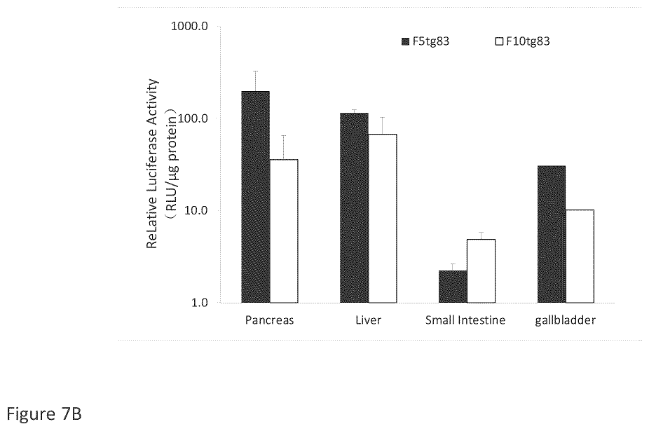

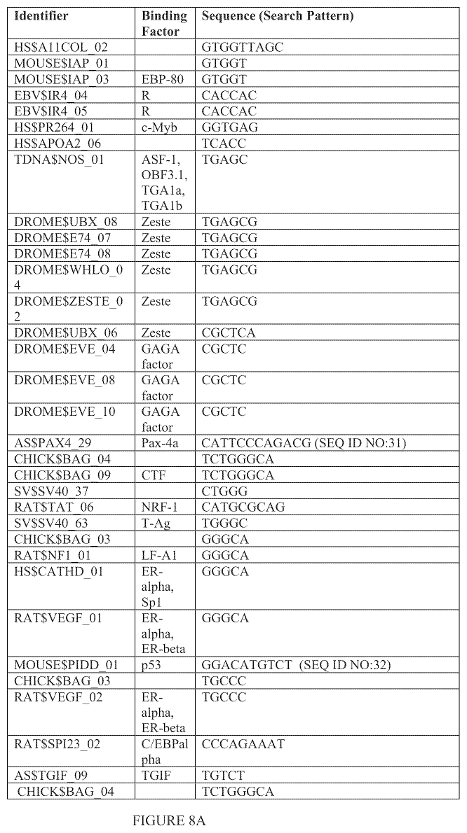

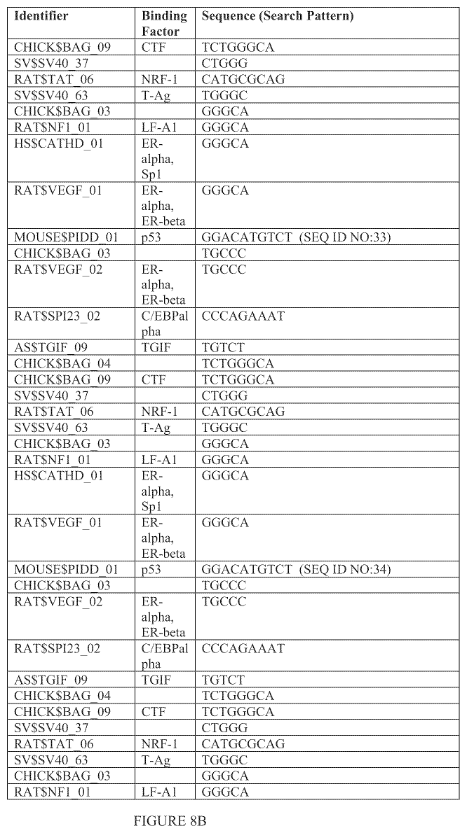

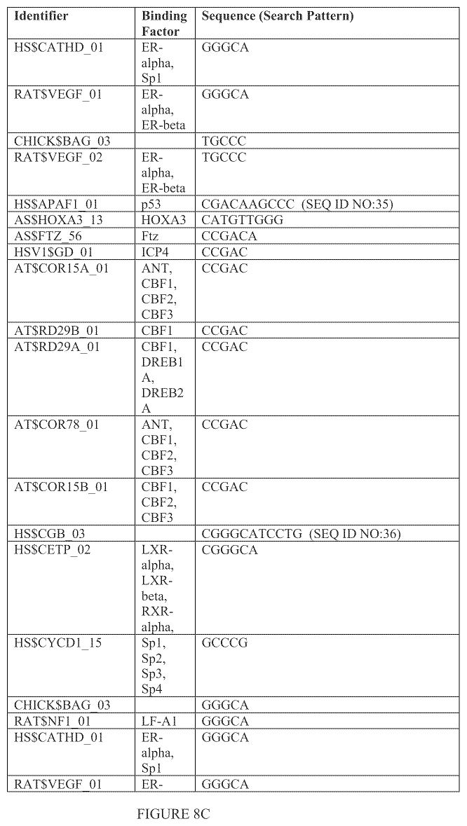

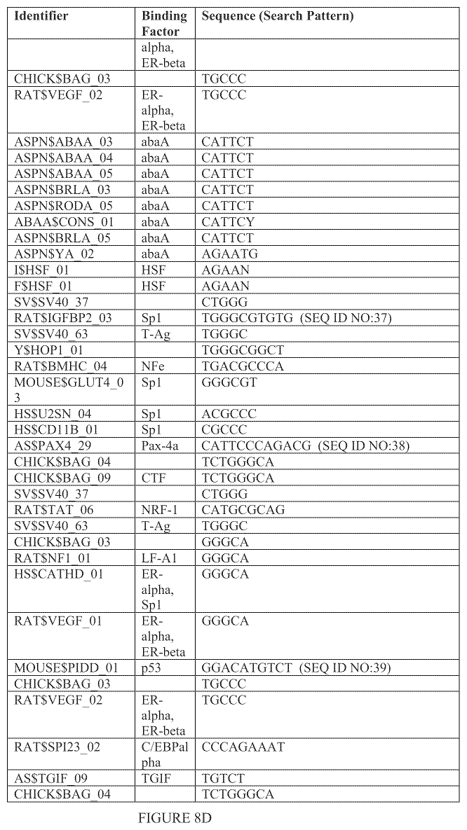

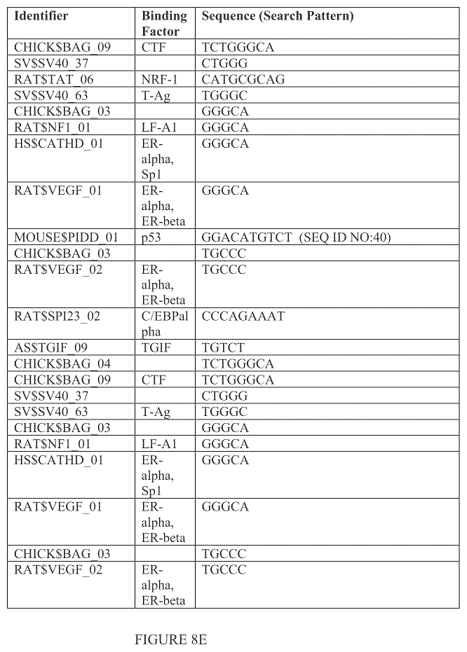

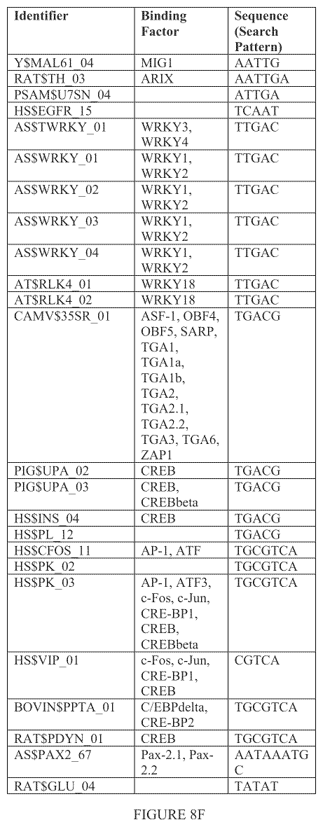

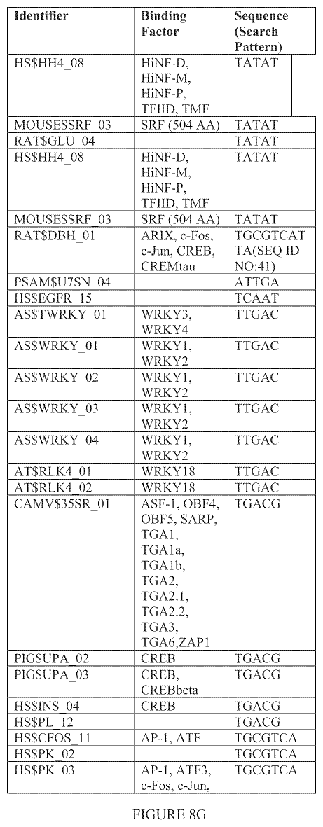

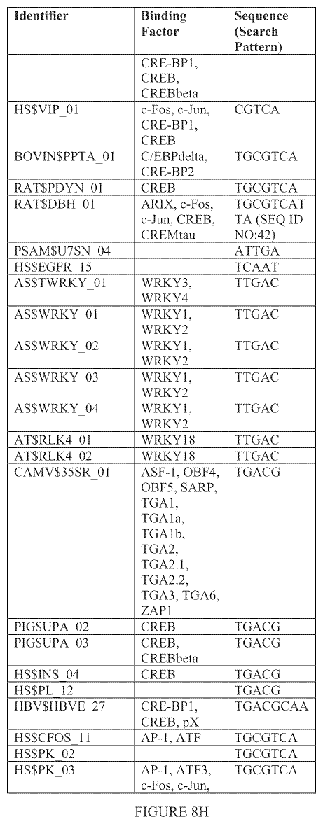

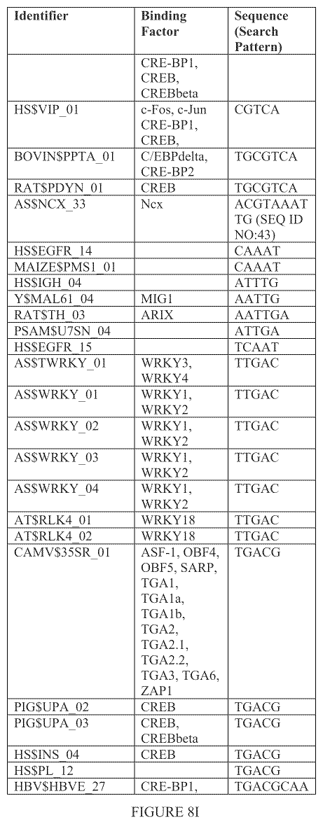

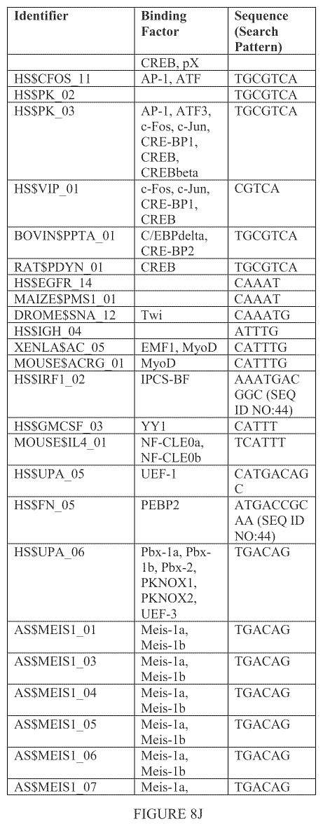

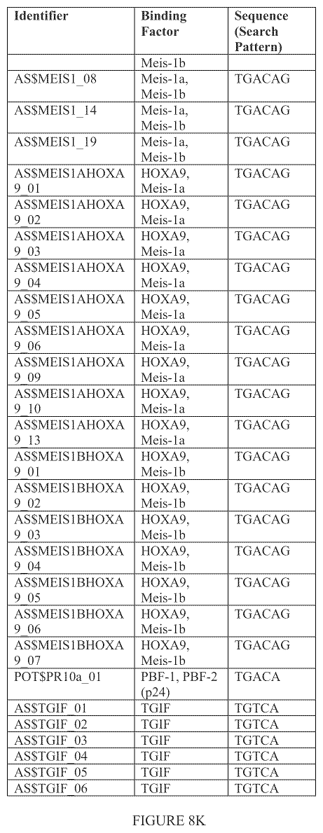

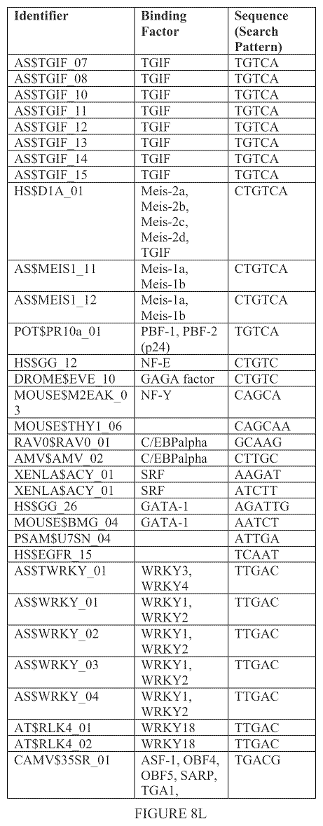

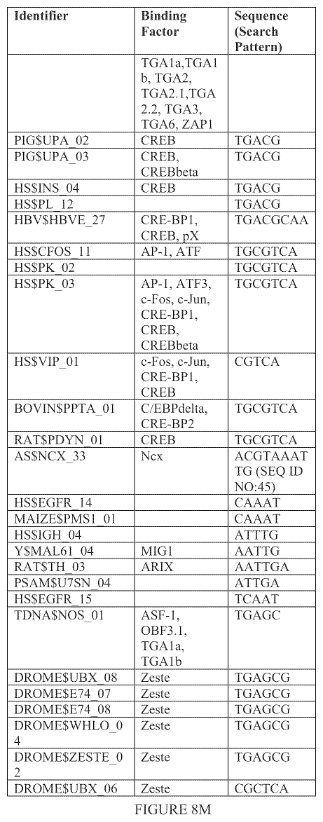

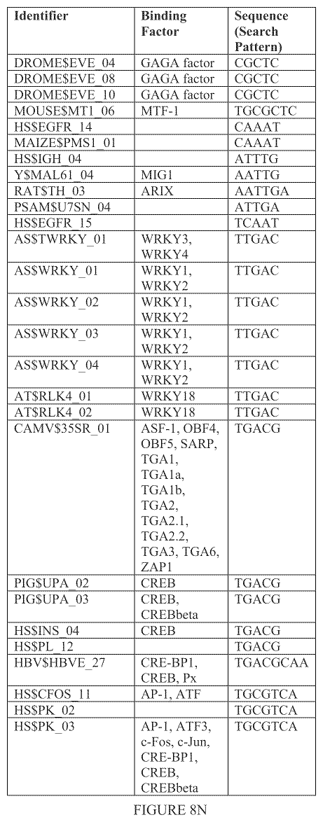

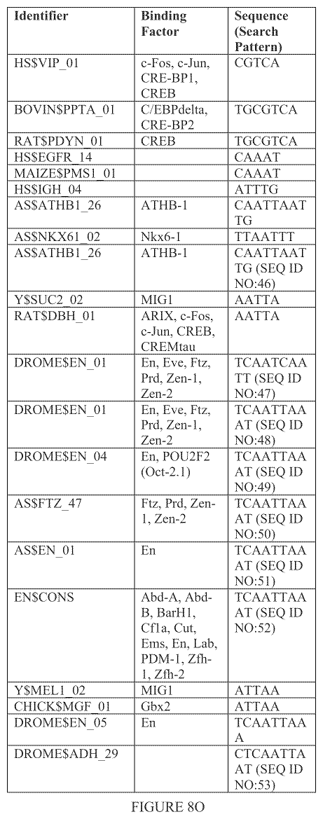

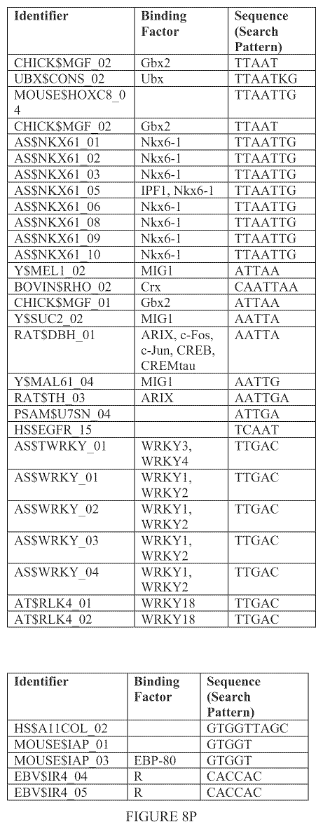

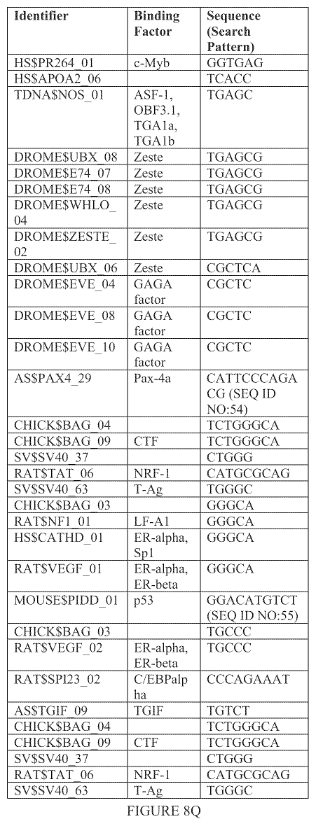

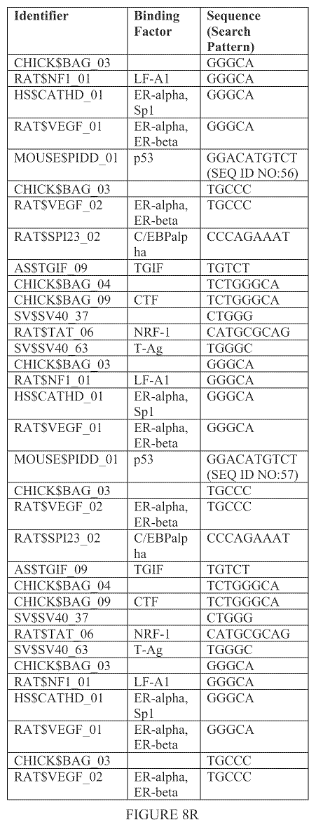

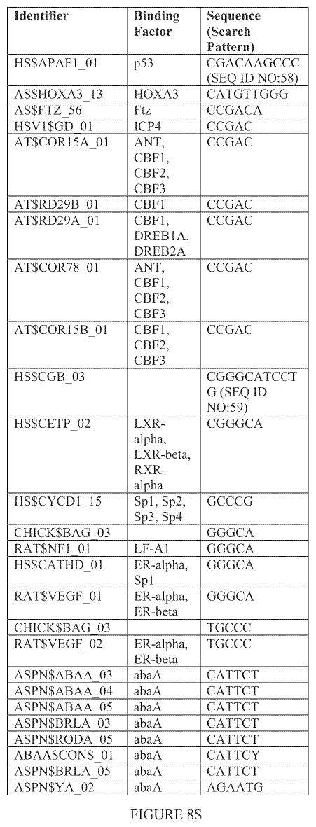

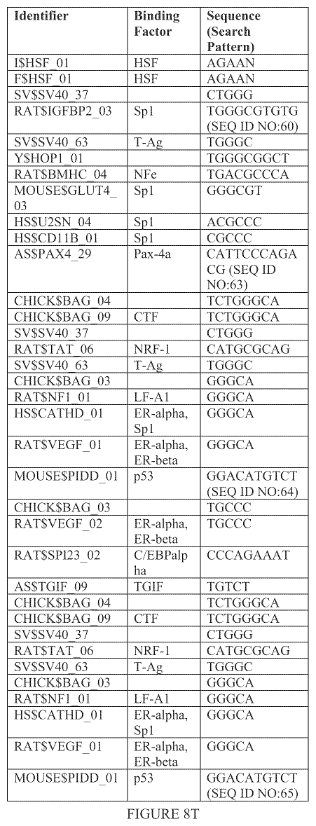

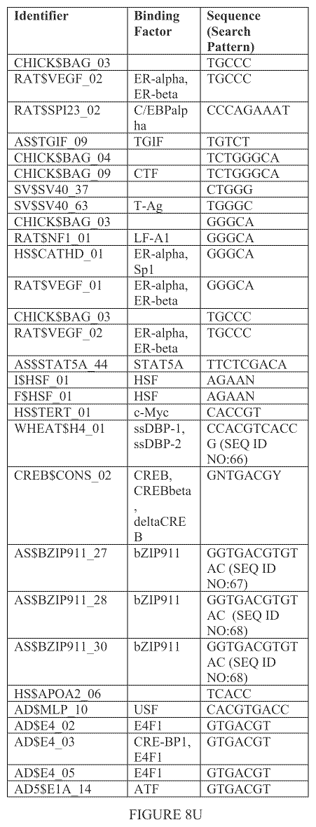

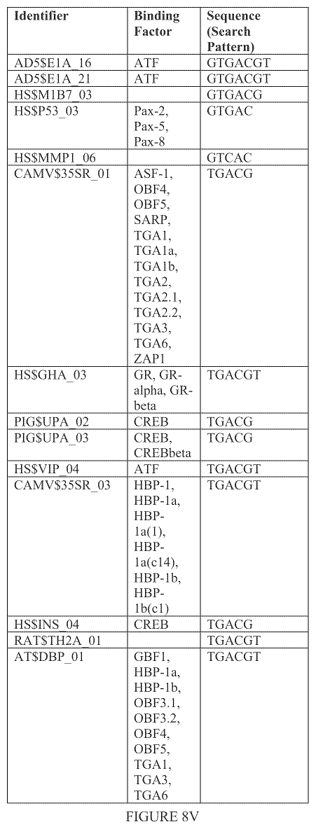

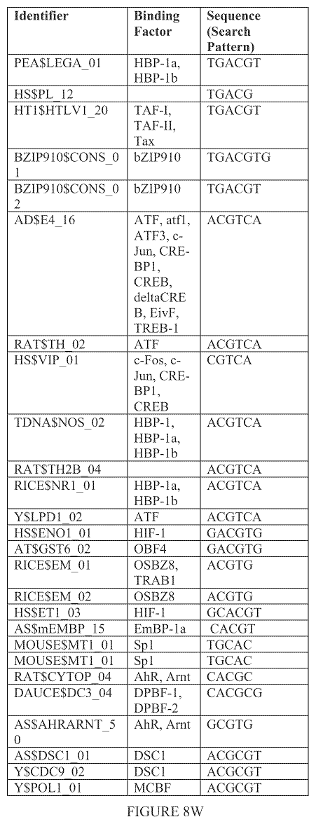

The enhancer/promoter combinations for lung epithelium (e.g., F5tg83) may not necessarily be as useful for other organs/tissue. For example, when AAV vectors harboring a luciferase reporter gene driven by F5tg83 or F10tg83 were used to infect different tissues/organs of the digestive system, F5tg83 demonstrated the strongest promoter activity in pancreas, gallbladder and liver, whereas F10tg83 outperformed F5tg83 in small intestine (FIGS. 7A-7B).

Although the studies were focused on identifying strong synthetic enhancer/promoter sequence using for efficiently expressing CFTR in lung/airway tissue, the screen approach can be used for any desired cell types, tissues and organs

### In Vitro and In Vivo.

Thus, the use of short enhancer elements (about 100-mer synthetic oligonucleotide sequences consisting of 10-mer repeats) was found to enhance gene expression from a minimal promoter in rAAV vectors. These 100 bp enhancer elements were previously identified for their ability to activate transcription directed by the CMV immediate-early (IE) minimal promoter in cell lines (Schlabach et al., 2010). It was hypothesized that enhancers that are most potent in activating transcription could be used to enhance activity of the synthetic tg83 promoter in airway cells in the context of rAAV vectors. Eight combinations of the tg83 promoter and 100 bp synthetic enhancers were tested, and one designated as F5 efficiently was found to enhance the transcription from the tg83 promoter in polarized airway cells (in vitro) derived from both humans and ferrets, as well as in the ferret airway (in vivo). Using the F5tg83 promoter and the ferret CFTR minigene of partial deletion at R domain (fCFTRΔR), a rAAV vector (AV2/2.F5tg83-fCFTRΔR) was found and its ability to correct CFTR-mediated C1-transport in CF ALI cultures tested.

### Example 2

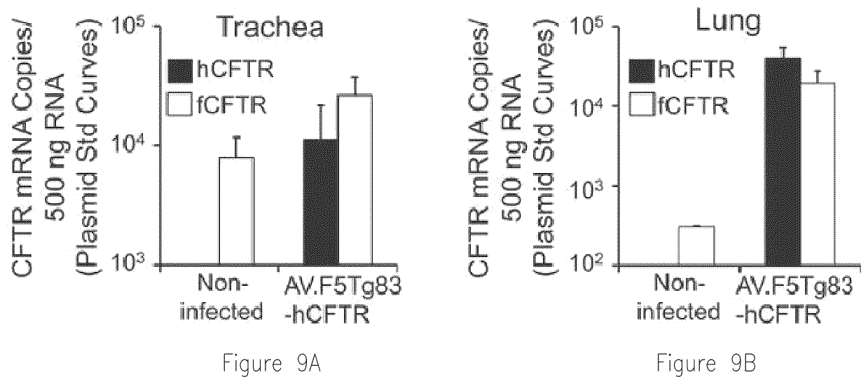

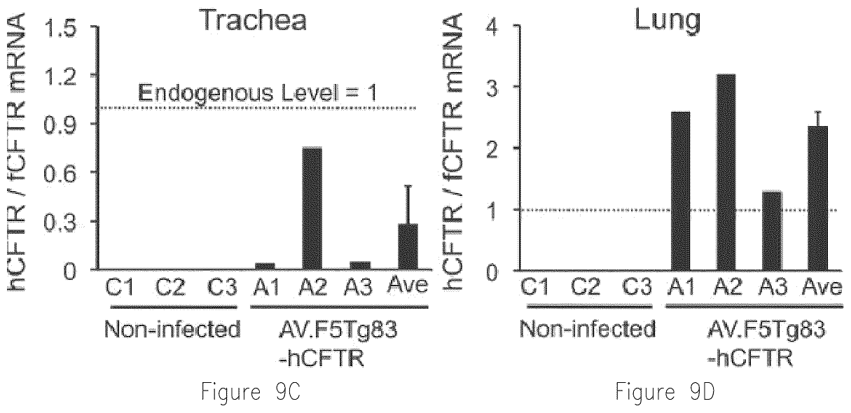

AV.F5Tg83-hCFTRΔR was tested for hCFTR expression in the newborn and mature ferret airway. An endpoint of these analyses was the ratio of transgene-derived human CFTR (hCFTR) to that of endogenous ferret CFTR (fCFTR) mRNA. In 3 day old newborn ferrets (FIG. 9), AV.F5Tg83-CFTRΔR led to 240% greater expression of human CFTR compared with endogenous (ferret) CFTR following gene delivery to the lung.

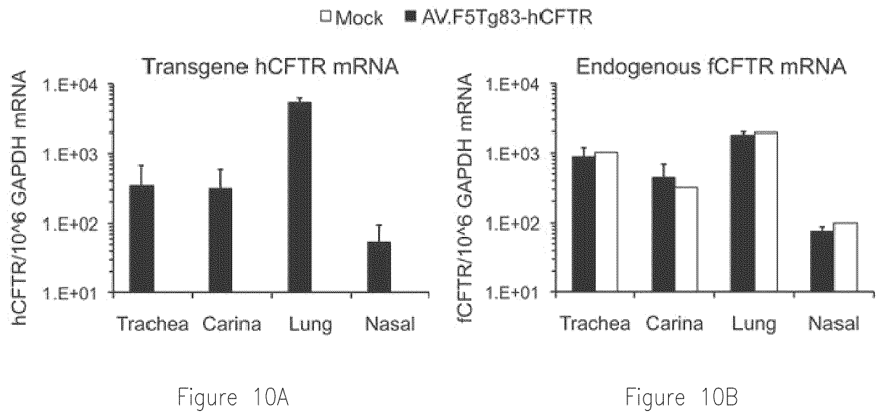

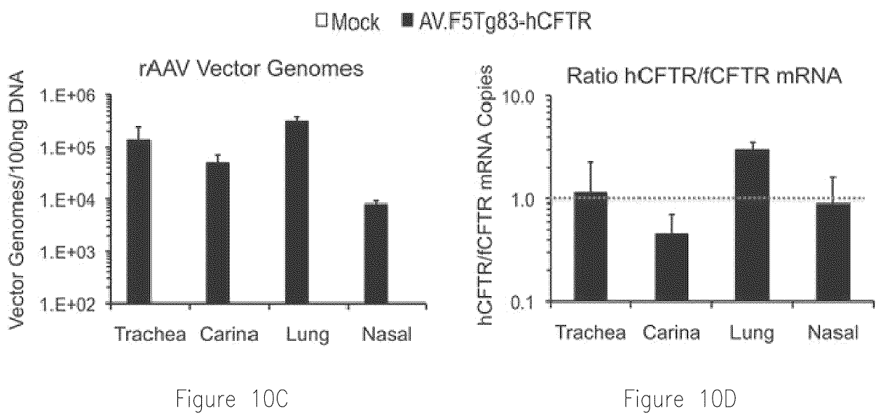

Given that the phenotype of ferret airway epithelia and the secretions in the airway change as the lungs mature, it was evaluated whether AV.F5Tg83-CFTRΔR transduces the mature ferret airway and the promoter remains active. To this end, the ability of AV.F5Tg83-CFTRΔR to transduce the lung of 1 month old ferrets was evaluated. In 1 month old mature ferrets (FIG. 10), AV.F5Tg83-CFTRΔR led to 300% greater expression of human CFTR compared with endogenous (ferret) CFTR following gene delivery to the lung. Furthermore, the ratio of human to ferret CFTR was approximately one in the nasal passage. These findings from newborn and mature ferrets suggest that the F5Tg83 promoter robustly expresses the CFTR transgene in the lung in vivo.

### REFERENCES

Aitken et al., Chest, 123:792 (2003).
Aitken et al., Hum Gene Ther., 12:1907 (2001).
Carter, Hum Gene Ther., 16:541 (2005).
Conrad et al., Gene Ther., 3:658 (1996).
Daya & Berns, Clin. Microb. Rev., 21:583 (2008).
Ding et al., Gene Ther., 12:873 (2005).
Ding et al., Mol. Ther., 12:671 (2006).
Dong et al., Hum. Gene Ther., 7:2101 (1996).
Duan et al., Hum. Gene Ther., 2:2761 (1998).
Duan et al., J. Clin. Invest., 105:1573 (2000).
Farmen et al., Am. J. Physiol. Lung Cell Mol. Physiol., 289:1123 (2005).
Fisher et al., J. Biol. Chem., 287:21673 (2012).
Flotte et al., J. Biol. Chem., 268:3781 (1993).
Flotte, Curr. Opin. Mol. Ther., 3: 497 (2001).
Flotte, et al., J. Biol. Chem., 268:3781 (1993).
Gerard et al., Gene Ther., 10:1744 (2003).
Glozman et al., J. Cell Biol., 184:847 (2009).
Griesenbach et al., Virus Adapt. Treat., 2:159, (2010).
Griesenback & Alton, Adv. Drug Deliv. Rev., 61:128 (2009).
Kapranov et al., Hum. Gene Ther., 23:46 (2012).
Karp et al., Methods Mol. Biol., 188:115 (2002).
Li et al., Mol. Ther., 17:2067 (2009).
Liu et al., Am. J. Respir. Cell Mol. Biol., 36:313 (2007).
Liu et al., Gene Ther., 14:1543 (2007).
Liu et al., Mol. Ther., 15:2114 (2007).
Moss et al., Hum. Gene Ther., 18:726 (2007).
Mueller & Flotte, Clin. Rev. Allergy Immunol., 35:164 (2008).
Olivier et al., J. Clin. Invest., 122:3755 (2012).
Ostedgaard et al., Proc. Natl. Acad. Sci. USA, 102:2952 (2005).
Ostedgaard et al., Proc. Natl. Acad. Sci. USA, 108:2921 (2011).
Ostedgaard et al., Proc. Natl. Acad. Sci. USA, 99:3093 (2002).
O'Sullivan & Freedman, Lancet, 373:1891 (2009).
Riordan et al., Science, 245:1068 (1989).
Rommens et al., Science, 245:1059 (1989).
Rowe et al., N. Engl. J. Med., 352:1992 (2005).
Schlabach et al., Proc. Natl. Acad. Sci. USA, 107:2538 (2010).
Summer-Jones et al., Gene Therapy for Cystic Fibrosis Lung Disease, Birkhauser Basel, Basel (2010).
Sun et al., Am. J. Pathol., 184:1309 (2014).
Sun et al., Am. J. Respir. Cell Mol. Biol., 50:502 (2014).
Sun et al., J. Clin. Invest., 118:1578 (2008).
Sun et al., J. Clin. Invest., 120:3149 (2010).
Wagner et al., Hum. Gene Ther., 13:1349 (2002).
Welsch, FASEB. J., 4:2718 (1990).
Welsh et al., In The Metabolic and Molecular Bases of Inherited Disease, eds. McGraw-Hill, New York, p. 3799 (1995).
Wu et al., Mol. Ther., 14:316 (2006).
Wu et al., Mol. Ther., 18:80 (2010).
Yan et al., Hum. Gene Ther., 24:786 (2013).
Yan et al., Hum. Gene Ther., 26:334 (2015).
Yan et al., J. Virol., 76:2043 (2002).
Yan et al., J. Virol., 78:2863 (2004).
Yan et al., Mol. Ther., 21:2181 (2013).
Zabner et al., Am J. Physiol. Lung Cell Mol. Physiol., 284:844 (2003).
Zhang et al., Mol. Ther., 10:990 (2004).
Zhang et al., Proc. Natl. Acad. Sci. USA, 95:10158 (1998).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method to express a transgene in a cell, comprising: introducing a composition comprising a recombinant parvovirus vector comprising a synthetic enhancer comprising a plurality of enhancer sequences operably linked to a promoter operably linked to a transgene to a eukaryotic cell so as to express the transgene in the cell.

2. The method of claim 1, wherein the cell is in a mammal.

3. The method of claim 1, wherein the expression of the transgene is enhanced by at least 2-, 5-, 10-, or 15-fold or more relative to a corresponding parvovirus vector that lacks the synthetic enhancer.

4. The method of claim 1, wherein the plurality of enhancer sequences has 2 up to 20, 2 up to 15 or 2 up to 10, of the enhancer sequences in tandem.

5. The method of claim 1, wherein the synthetic enhancer comprises F1, F5 or F10, the synthetic enhancer has at least one enhancer sequence with at least 80% nucleotide sequence identity to F1, F5 or F10, the synthetic enhancer has at least one enhancer sequence with at least 90% nucleotide sequence identity to F1, F5 or F10 or the synthetic enhancer has at least one enhancer sequence with at least 95% nucleotide sequence identity to F1, F5 or F10.

6. The method of claim 1, wherein the synthetic enhancer and promoter have at least 80%, 85%, 90%, 95%, 98%, 99% or more nucleotide sequence identity to one of SEQ ID NOs. 24-30.

7. The method of claim 1, wherein the synthetic enhancer and promoter have a sequence having at least 90% nucleotide sequence identity to SEQ ID NO:27.

8. The method of claim 7, wherein the synthetic enhancer and promoter have at least 95%, 98%, 99%, or more nucleotide sequence identity to SEQ ID NO:27.

9. The method of claim 8, wherein the synthetic enhancer and promoter comprise the sequence of SEQ ID NO:27.

10. The method of claim 1, wherein the recombinant parvovirus vector is a bocavirus or an adeno-associated virus vector.

11. The method of claim 10, wherein the recombinant parvovirus vector is an adeno-associated virus vector.

12. The method of claim 1, wherein the promoter is operably linked to an open reading frame that encodes a prophylactic or therapeutic gene product.

13. The method of claim 12, wherein the therapeutic gene product is cystic fibrosis transmembrane conductance regulator (CFTR) or CFTRΔR.

14. The method of claim 13, wherein the CFTR or CFTRΔR is human CFTR or human CFTRΔR.

15. The method of claim 14, wherein the CFTRΔR is human CFTRΔR.

16. A method to express a human CFTRΔR transgene in a cell, comprising: introducing a composition comprising a recombinant parvovirus vector to a eukaryotic cell so as to express the human CFTRΔR transgene in the cell, wherein the recombinant parvovirus vector comprises (i) an enhancer operably linked to a promoter, wherein the enhancer and promoter comprise the sequence of SEQ ID NO.:27; and (ii) the human CFTRΔR transgene operably linked to the promoter.
